(19) United States
(12) Patent Application Publication
(10) Pub. No.: US 2026/0284360 A1
(43) Pub. Date: Sep. 24, 2026

(54) SYSTEMS AND METHODS FOR TRANSCATHETER AORTIC VALVE TREATMENT

(71) Applicant: Silk Road Medical, Inc., Sunnyvale, CA (US)

(72) Inventors: Kristen Lipschultz, Sunnyvale, CA (US); Brad Steele, Sunnyvale, CA (US); Zach Tegels, Sunnyvale, CA (US); Karl Halden, Sunnyvale, CA (US)

(73) Assignee: Silk Road Medical, Inc., Sunnyvale, CA (US)

(21) Appl. No.: 18/874,141

(22) PCT Filed: Jun. 14, 2023
(86) PCT No.: PCT/US2023/025266

§ 371 Date: Dec. 12, 2024

Related U.S. Application Data
(60) Provisional application No. 63/352,143, filed on Jun. 14, 2022.
(60) Provisional application No. 63/486,341, filed on Feb. 22, 2023.

(51) Int. Cl. A61M 25/06; A61F 2/24; A61M 25/00

(52) U.S. Cl. CPC A61M 25/0662; A61F 2/2433; A61M 25/0068; A61M 2025/0006; A61M 2202/0021; A61M 2202/0028; A61M 2202/0413; A61M 2202/06; A61M 2205/0266; A61M 2205/10; A61M 2205/7545; A61M 2210/127

(57) ABSTRACT

A system is configured for treating an artery. The system includes an outer delivery sheath configured to be delivered to a vascular location in communication with an aortic arch, and an inner catheter defining an inner lumen. An outer lumen is formed between an outer wall of the inner catheter and an inner wall of the outer delivery sheath and a filter is positioned at a distal end of the inner catheter. An extracorporeal shunt is configured to receive blood flow from the inner lumen and a filter is coupled to the shunt.

### CROSS-REFERENCE TO RELATED APPLICATIONS

The current application claims priority under 35 U.S.C. § 119(e) to U.S. Provisional patent application Ser. Nos. 63/352,143, filed on Jun. 14, 2022, and entitled “SYSTEMS AND METHODS FOR TRANSCATHETER AORTIC VALVE TREATMENT,” and 63/486,341, filed on Feb. 22, 2023, and entitled “SYSTEMS AND METHODS FOR TRANSCATHETER AORTIC VALVE TREATMENT,” which are incorporated by reference herein in their entirety.

### BACKGROUND

The present disclosure relates to methods and devices for replacing heart valves.

Patients with defective aortic heart valves are often candidates for a replacement heart valve procedure. The conventional treatment is the surgical replacement of the heart valve with a prosthetic valve. This surgery involves a gross thorocotomy or median sternotomy, cardiopulmonary bypass and cardiac arrest, surgical access and excision of the diseased heart valve, and replacement of the heart valve with a provided good long term outcomes for these patients, with durability of up to ten or fifteen years for tissue valves, and even longer for mechanical valves. However, heart valve replacement surgery is highly invasive, can require lengthy recovery time, and is associated with short and long term complications. For high surgical risk or inoperable patients, this procedure may not be an option.

A minimally invasive approach to heart valve replacement has been developed. This approach, known as transcatheter aortic valve implantation (TAVI) or replacement (TAVR), relies on the development of a collapsible prosthetic valve which is mounted onto a catheter-based delivery system. This type of prosthesis can be inserted into the patient through a relatively small incision or vascular access site, and may be implanted in the beating heart without cardiac arrest. The advantages of this approach include less surgical trauma, faster recovery time, and lower complication rates. For high surgical risk or inoperable patients, this approach offers a good alternative to conventional surgery.

### SUMMARY

There is a need for an access system for endovascular prosthetic aortic valve implantation that provides a generally shorter and straighter access path than current systems and methods. There is also a need for an access system that provides protection from cerebral embolic complications during the procedure.

Disclosed herein are devices and methods that allow transcarotid or subclavian access via the common carotid artery to the native aortic valve, and transcatheter implantation of a prosthetic aortic valve into the heart or aorta. The devices and methods also provide means for embolic protection during such an endovascular aortic valve implantation procedure.

In one aspect, there is disclosed a system for treating an artery, comprising: an outer delivery sheath, the outer delivery sheath configured to be delivered to a vascular location in communication with an aortic arch; an inner catheter defining an inner lumen, the inner catheter slidably positioned inside the outer delivery sheath such that an outer lumen is formed between an outer wall of the inner catheter and an inner wall of the outer delivery sheath; a filter positioned at a distal end of the inner catheter, the filter configured to guide debris from a blood vessel into the inner lumen of the inner catheter via blood flow into the filter; and an extracorporeal shunt configured to receive blood flow from the inner lumen; and a filter coupled to the shunt.

Other aspects, features and advantages should be apparent from the following description of various embodiments, which illustrate, by way of example, the principles of the disclosure.

### BRIEF DESCRIPTION OF THE DRAWINGS

FIG. 1 shows a side view of an exemplary access sheath having an occlusion element mounted on the sheath.

FIG. 2 shows a side view of an exemplary access sheath having a filter element mounted on the sheath.

FIG. 3 shows a front view of the filter element.

FIGS. 4, 5A, and 5B show alternate embodiments of the access sheath.

FIG. 6 schematically depicts a view of the vasculature showing normal circulation.

FIGS. 7A and 7B shows other embodiments of an access sheath deployed in the vasculature.

FIGS. 8A and 8B shows other embodiments of an access sheath deployed in the vasculature.

FIG. 9 shows an embodiment of a transcarotid prosthetic aortic valve and delivery system.

FIG. 10 shows an alternate embodiment of a transcarotid prosthetic aortic valve and delivery system.

FIG. 11 shows another embodiment of an access sheath deployed in the vasculature with an occlusion element occluding the left common carotid artery.

FIG. 12 shows another embodiment of an access sheath deployed in the vasculature with an occlusion element occluding the right common carotid artery.

FIG. 13 shows another embodiment of an access sheath deployed in the vasculature with an occlusion element occluding the innominate artery.

FIG. 14 shows a delivery system deploying an endovascular prosthetic valve via an access sheath 110 and guidewire 119.

FIG. 15A shows a schematic representation of an embodiment wherein an access sheath is used to gain transcervical access to a vessel.

FIG. 15B shows a schematic view of the vasculature with a catheter positioned to access the aortic arch.

FIG. 15C shows a schematic view of the vasculature with a catheter positioned via the LCCA so as to be able to inject fluid into the aortic arch.

FIG. 15D shows a schematic view of the vasculature with a catheter (or sheath) inserted into the right common carotid artery to access the aortic arch.

FIG. 15E shows a schematic view of a distal region of the catheter positioned in the aortic arch.

FIG. 16A shows a schematic representation of another embodiment wherein an arterial shunt is combined with or otherwise coupled to an access sheath.

FIG. 16B shows a schematic representation of possible flow routes that can be achieved by use of a shunt.

FIG. 17 shows an embodiment wherein a balloon is placed in the contralateral CCA.

FIGS. 18A through 18C show additional embodiments wherein one or more occlusion balloon catheters are used.

FIG. 19 shows an embodiment wherein blood is pulled through an aspiration catheter placed in the contralateral CCA.

FIG. 20 shows an embodiment wherein a vascular valve is placed in the contralateral CCA.

FIG. 21A shows a schematic representation of an embodiment of a conduit and shunt system.

FIG. 21B shows a schematic representation of a distal region of a conduit with a distal region of a secondary catheter providing a pathway extending into the blood vessel.

FIG. 22A shows an embodiment of a distal region of a catheter device configured to capture embolic debris from other devices and/or the patient's vasculature.

FIG. 22B shows the device of FIG. 22A in cross section.

FIG. 23 shows a neuroprotection sheath deployed in the vasculature.

FIG. 24 shows a transparent view of a sheath.

FIG. 25 shows a cross-sectional view of the sheath of FIG. 24.

FIG. 26 schematically depicts a view of the vasculature showing normal antegrade circulation.

FIG. 27A shows a schematic, side view representation of a distal region of a filter system.

FIG. 27B shows another embodiment of the filter system.

FIG. 28 shows a schematic representation of the filter system.

FIG. 29 shows a schematic representation of the filter system in cross section.

FIG. 30 shows a schematic representation of the vasculature wherein a filter has been deployed in the aortic arch via a delivery sheath.

FIG. 31 schematically shows a filter basket.

FIG. 32 shows an embodiment wherein one or more filters are deployed to the blood vessels branching off of the aortic arch.

### DETAILED DESCRIPTION

Disclosed herein are devices and methods that allow arterial access, such as transcarotid access via the common carotid artery, or subclavian access via the subclavian artery to the native aortic valve, and implantation of a prosthetic aortic valve into the heart or aorta. The devices and methods also provide means for embolic protection during such an endovascular aortic valve implantation procedure. It should be appreciated that the disclosed systems and methods can also be configured for use via a transfemoral access location and are not limited to use via a specific access location.

In an embodiment, transcarotid or subclavian access to the aortic valve is accomplished via either a percutaneous puncture or direct cut-down to the artery. A cut-down may be advantageous due to the difficulty of percutaneous vessel closure of larger arteriotomies in the common carotid artery. If desired, a pre-stitch may be placed at the arteriotomy site to facilitate closure at the conclusion of the procedure. An access sheath with associated dilator and guidewire is provided which is sized to fit into the common carotid or subclavian artery. The access sheath is inserted into the artery inferiorly towards the aortic arch. Either the left or the right common carotid or subclavian artery may be selected as the access site, based on factors including, for example, the disease state of the proximal artery and/or the aorta and the angle of entry of the carotid or innominate artery into the aorta. The carotid artery may then be occluded distal to the access site. If the access is via a direct surgical cut-down and arteriotomy, the occlusion may be accomplished via a vascular clamp, vessel loop, or Rummel tourniquet. Alternately, the access sheath itself may include an occlusion element adapted to occlude the artery, for example an occlusion balloon, to prevent embolic particulates from entering the carotid artery distal to the access site during the procedure.

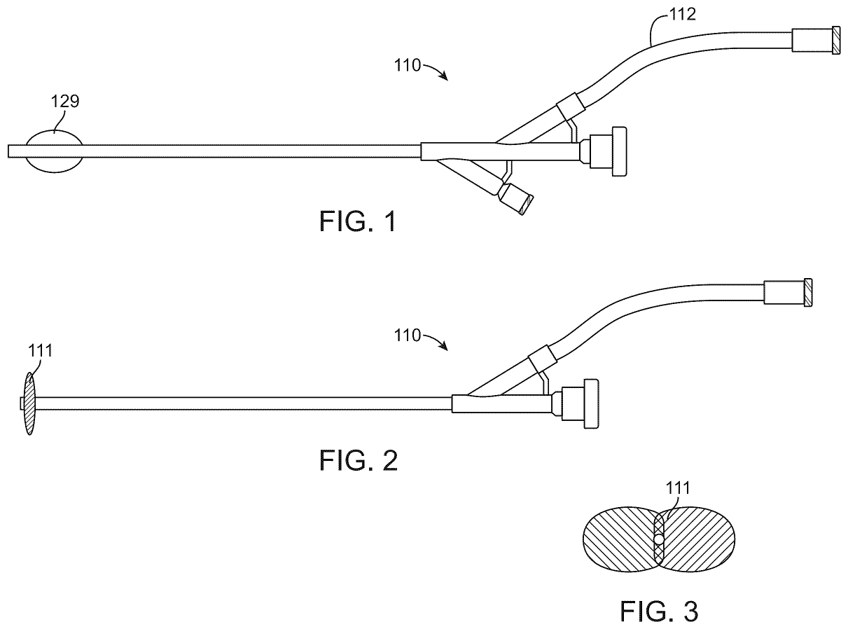

FIG. 1 shows a side view of an exemplary arterial access sheath 110 formed of an elongate body having an internal lumen. In an embodiment, the sheath has a working length of 10-60 cm wherein the working length is the portion of the sheath that is insertable into the artery during use. The lumen of the sheath has an inner diameter large enough to accommodate insertion of an endovascular valve delivery system, such as an 18 French to 22 French (0.236″ to 0.288″) system. In an embodiment, the delivery system has an inner diameter as low as about 0.182″ The access sheath 110 can have an expandable occlusion element 129 positioned on the access sheath. The occlusion element 129 is configured to be expanded to a size for occluding flow through the artery. The occlusion element 129 may be placed anywhere in the artery or aorta. In an embodiment, the occlusion element is an occlusion balloon.

Once the sheath 110 is positioned in the artery, the occlusion element 129, if present, is optionally expanded within the artery to occlude the artery and possibly anchor the sheath into position. The arterial access sheath 110 may include a Y-arm for delivery of contrast or saline flush, for aspiration, and/or may be fluidly connected to a shunt, wherein the shunt provides a shunt lumen or pathway for blood to flow from the arterial access sheath 110 to a return site such as a vascular return site or a collection reservoir. In this regard, a retrograde or reverse blood flow state may be established in at least a portion of the artery. The sheath 110 may also include a Y-arm for inflation of the occlusion balloon via an inflation lumen, and a hemostasis valve for introduction of an endovascular valve delivery system into the sheath. Alternately, the sheath 110 may include an actuating element if the occlusion element is a mechanical occlusion structure. The endovascular valve delivery system may include a prosthetic valve and a delivery catheter. In an embodiment, the delivery catheter has a working length of 30-80-cm including 30, 40, 60, 70, or 80 cm although the length can vary.

In an embodiment, aspiration may be applied to the artery via the access sheath 110. In this regard, the access sheath 110 can be connected via a Y-arm 112 to an aspiration source, so that embolic debris may be captured which may otherwise enter the remaining head and neck vessels, or travel downstream to lodge into peripheral vessels. The aspiration source may be active, for example a cardiotomy suction source, a pump, or a syringe. Alternately, a passive flow condition may be established, for example, by fluidly connecting the Y-arm 112 to a shunt, which in turn is connected to a lower-pressure source such as a collection reservoir at atmospheric or negative pressure, or a venous return site in the patient. The passive flow rate may be regulated, for example, by controlling the restriction of the flow path in the shunt.

In an embodiment, the access system may be equipped with one or more embolic protection elements to provide embolic protection for one or both carotid arteries as well as one or all head or neck blood vessels. For example, a filter may be included in the access system to provide embolic protection for one or both carotid arteries. In a variation of this embodiment, the filter is deployed via the contralateral carotid, brachial or subclavian artery, and positioned in the aortic arch across the ostium. If the sheath access site is the left common carotid artery, the filter may be positioned across the ostium of the innominate (also known as brachiocephalic) artery. If the sheath access site is the right common carotid artery, the filter may be positioned across the ostium of the left common carotid artery. In a variation of this embodiment, the filter is deployed across both the innominate and left common carotid artery, or across all three head and neck vessels (innominate artery, left common carotid artery, and left subclavian artery). The filter element may be built-in to the access sheath 110. Or, the filter element may be a separate element which is compatible with the access sheath 110. For example, the filter element may be a coaxial element which is slideably connected to the access sheath or an element which is placed side-by-side with the access sheath. The filter element may comprise an expandable frame, so that it may be inserted into the artery in a collapsed state, but then expanded at the target site to position the filter element across the opening of the artery or arteries.

FIG. 2 shows a side view of an exemplary access sheath 110 having a filter element 111 mounted on the sheath. FIG. 3 shows a front view of the filter element 111 showing an exemplary profile of the filter element 111. In the embodiment of FIG. 3, the filter element 111 is sized and shaped to fit within and block the head and neck vessels. In an embodiment, the deployed filter has a long dimension of about 2, 3, 4, or 5 cm and a short dimension of about 1, 1.5, or 2 cm. The profile shown in FIG. 3 is for example and it should be appreciated that the shape of the filter element 111 may vary. For example, the shape of the filter element may be oval, round, elliptical, or rectangular. The filter material may be woven or knitted textile material, or may be a perforated polymer membrane such as polyurethethane. The filter material can also be Nitinol. The filter porosity may be 50, 100, 150, 200, or 300 microns, or any porosity in between. The expandable frame of the filter element may be made from spring material such as stainless steel or nitinol wire or ribbon.

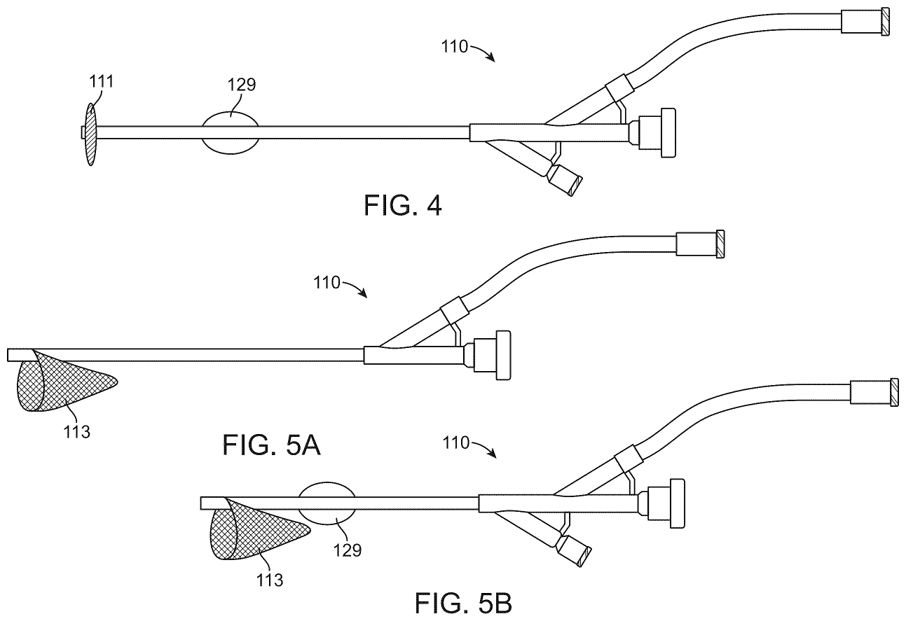

In the embodiment with the filter element, occlusion and/or aspiration means may still be part of the system, to provide embolic protection during filter deployment before the valve implantation and filter retrieval after valve implantation. The filter element itself may be a primary method of embolic protection during the implantation procedure. The sheath 110 may also be equipped with both an occlusion element 129 and a filter element 111, as shown in FIG. 4.

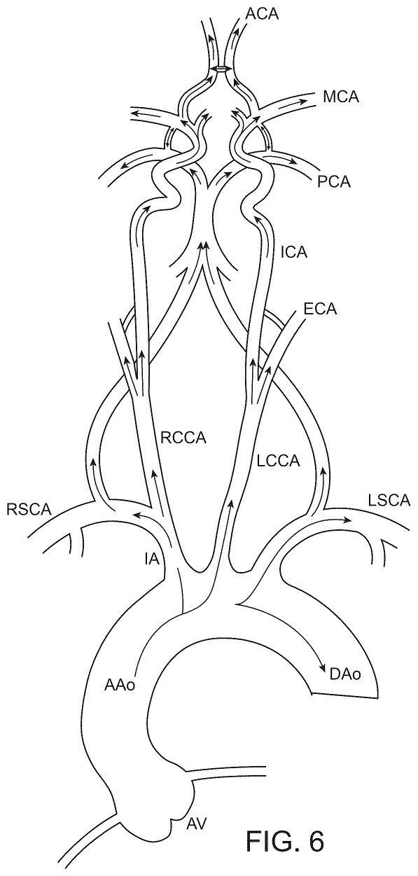

In another variation of this embodiment, shown in FIG. 5A, the sheath 110 includes an aortic filter element 113 which is sized and shaped to be deployed across the ascending aorta and thus protect all the head and neck vessels from embolic debris. The shape of the filter element may vary. In an embodiment, the shape of the filter element may be a cone or a closed-end tube. The expandable frame of the filter frame is sized and shaped to traverse the entire diameter of the aorta when deployed. For example the expandable frame may be a loop which can expand from 12 to 30 mm in diameter. Alternately, the expandable frame may be a series of struts connected at one or both ends and which expand outwardly to deploy the filter element across the diameter of the aorta. The filter material may be woven or knitted textile material, or may be a perforated polymer membrane such as polyurethethane. The filter porosity may be about 40, 100, 150, 200, or 300 microns, or any porosity in between.

The expandable frame of the filter element may be made from spring material such as stainless steel or nitinol wire or ribbon. As with the previous variation, occlusion and aspiration means may be included in this variation to provide protection during filter deployment and filter retrieval. The aortic filter element 113 may be integral to the sheath, or be a separate device which is compatible with the sheath, for example may be coaxial or side-by-side with the access sheath. As shown in FIG. 5B, an embodiment of the sheath 110 may include both an aortic filter element 113 and an occlusion element 129.

FIG. 6 schematically depicts a view of the vasculature showing normal antegrade circulation. The blood vessels are labeled as follows in FIG. 6: ACA: anterior cerebral artery; MCA: middle cerebral artery; PCA: posterior cerebral artery; ICA: internal carotid artery; ECA: external carotid artery; LCCA: left common carotid artery; RCCA: right common carotid artery; LSCA: left subclavian artery; RSCA: right subclavian artery; IA: innominate artery; AAo: Ascending aorta; DAo: descending aorta; AV: aortic valve.

In certain situations, it may be desirable to provide a mechanism for perfusing the carotid artery upstream of the entry point of the access sheath 110 into the carotid or innominate artery. If the access sheath 110 is similar in size to the carotid or innominate artery, flow through the artery may be essentially blocked by the access sheath when the sheath is inserted into the artery. In this situation, the upstream cerebral vessels may not be adequately perfused due to blockage of the carotid artery by the sheath. In an embodiment of the access sheath 110, the sheath includes a mechanism to perfuse the upstream carotid and cerebral vessels.

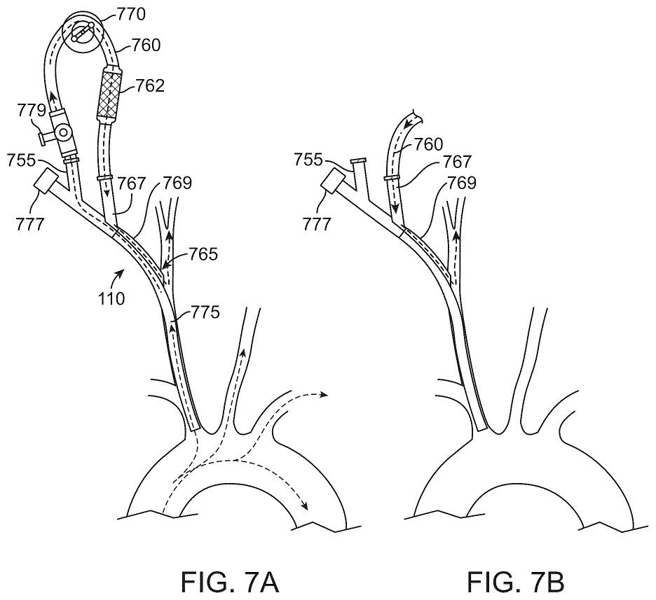

FIG. 7A shows an exemplary embodiment of such an access sheath 110 deployed in the vasculature. A proximal portion of the access sheath has two parallel, internal lumens that are part of a single monolithic structure of the access sheath. A first lumen 775 extends from the proximal end of the sheath to the distal tip of the sheath and is fluidly connected on the proximal end to a shunt Y-arm 755 and a hemostasis valve 777 located at the proximal end of the sheath. The first lumen 775 is sized and shaped to receive and enable delivery of a transcatheter aortic valve and delivery system via the hemostasis valve 777. For example, the first lumen has a length such that its distal opening is positioned at the heart or aorta. A second lumen 769 is positioned adjacent the first lumen and extends from the proximal end of the sheath to a distal opening at a location mid-shaft 765 and is fluidly connected on the proximal end to a second, perfusion Y-arm 767. There is an opening on the distal end of the second lumen at the location 765. The second lumen 769 is sized and shaped to enable shunting of blood to the carotid artery distal of the access sheath insertion site. A radiopaque shaft marker may be positioned on the sheath at this location to facilitate visualization of this opening to the user under fluoroscopy. The perfusion lumen has a length such that the distal opening of the perfusion lumen can be positioned in and perfuse a distal carotid artery when in use. The proximal end of the first lumen has a proximal connector with the hemostasis valve 777 and a Y-arm. As mentioned, the hemostasis valve is sized to fit therethrough an arterial valve delivery system. The proximal end of the perfusion lumen also has a proximal connector. The proximal connectors and/or Y-arms permit a shunt to be attached.

The Y-arm 755 is removably connected to a flow shunt 760 which in turn is removably connected to the second Y-arm 767. The shunt defines an internal shunt lumen that fluidly connects the first lumen 775 to the second lumen 769. A stopcock 779 may be positioned between the Y-arm 755 and the flow shunt 760 to allow flushing and contrast injection while the shunt 760 is connected. When the sheath is positioned in the artery, arterial pressure drives blood flow into the distal end of the first lumen 775 of the arterial access sheath, out the first lumen from Y-arm 755, then into the shunt 760, and back into the sheath via the Y-arm 767. The blood then flows into the parallel lumen 769 and into the distal carotid artery at the location 765 to perfuse the vasculature distal of the arterial sheath 110. An in-line filter element 762 may be included in the flow shunt 760 so that emboli generated during the procedure are not perfused into the cerebral artery. In the event the sheath 110, shunt 760, and lumen 769 create a flow restriction that limits adequate perfusion, the flow shunt 760 may incorporate an active pump 770 to drive blood flow and provide the required level of cerebral perfusion. This may be especially true when the valve is being delivered through the first lumen 775 of the access sheath 110.

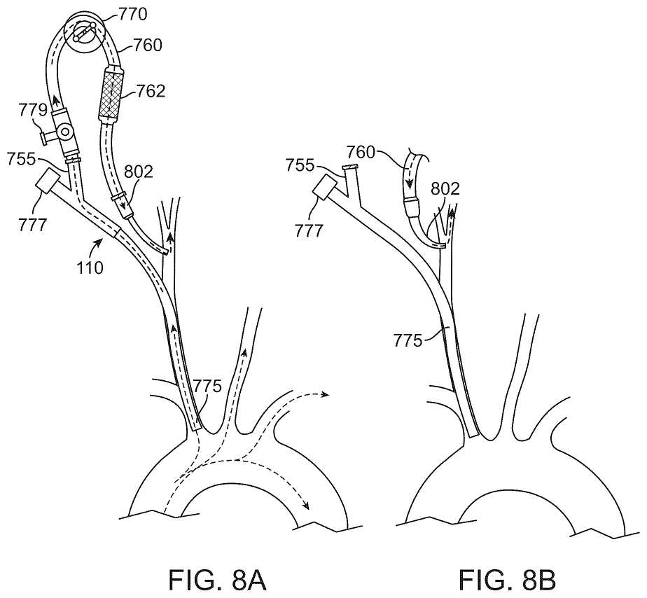

FIG. 7B shows a variation of the embodiment of FIG. 7A. The Y-arm 767 fluidly connects the shunt 760 to the parallel lumen 769 that re-introduces blood from the shunt 760 into the artery at location 765 when positioned in the artery. The shunt 760 in this embodiment is not fluidly connected to the first lumen 775 in the sheath. The shunt 760 rather than receiving blood from the access sheath via Y-arm 755 may be connected to another arterial blood source via a second sheath, for example a femoral or subclavian artery or the contralateral carotid artery. In this variation, the shunted blood flow is not restricted by the delivery of the valve through the first lumen 775 of the access sheath. In this embodiment, there is no need for a filter 762 in the shunt line, as the blood source is far from the treatment area and there is minimal risk of distal emboli in the shunted blood. The Y-arm 755 may still be used for flushing and contrast injection into the sheath. In another variation, shown in FIG. 8A, the arterial access sheath 110 has a single lumen 775 which is fluidly connected to a Y-arm 755 at the proximal region. The lumen 775 is sized and shaped to receive and enable delivery of a transcatheter aortic valve and delivery system via a hemostasis valve 777. The Y-arm 755 is connected to a flow shunt 760 which in turn is connected to a second arterial sheath 802 which is sized and shaped to be introduced into the carotid artery distal to arterial access point where the access sheath 110 is introduced. A stopcock 779 may be positioned between the Y-arm 755 and the flow shunt 760 to allow flushing and contrast injection while the shunt 760 is connected. When the sheath is properly positioned in the artery, the arterial pressure drives flow into the lumen 775 of the sheath 110, out the first lumen via Y-arm 755, then through the shunt 760, through the second catheter 802, and back into the carotid artery upstream from the arterial access point to perfuse the vasculature distal of the arterial sheath 110. As above, a filter element 762 may be included in the flow shunt 760 so that emboli generated during the procedure are not perfused into the cerebral artery. In the event the sheath 110 and shunt 760 experience flow restriction that limits adequate perfusion, for example when the valve is being delivered through the lumen 775 of the access sheath 110, the flow shunt may incorporate an active pump 770 to drive blood flow and provide the required level of cerebral perfusion.

FIG. 8B shows a variation of the embodiment of FIG. 8A. Here, the second arterial sheath 802 is removably or fixedly connected to a shunt or flow line 760 which in turn is connected to another arterial source via another sheath, for example a femoral or subclavian artery or the contralateral carotid artery. In this variation, the shunted blood flow is not restricted by the delivery of the valve through the lumen 775 of the access sheath. In this embodiment, there is no need for a filter 762 in the shunt line, as the blood source is far from the treatment area and there is minimal risk of distal emboli in the shunted blood. The Y-arm 755 may still be used for flushing and contrast injection into the sheath.

In the embodiments described above in FIGS. 7A and 7B and FIGS. 8A and 8B, the arterial access sheath 110 may include an occlusion element (not shown) at the distal end of the sheath, configured to occlude the carotid artery and to assist in prevention of emboli from entering the carotid artery. Additionally, in these embodiments, the flow shunt 760, and if applicable pump 770 and/or the second sheath 820 may be provided as separate components in a single kit to enable transcarotid access and carotid shunting during a catheter-based aortic valve replacement procedure.

Although the figures show sheath insertion in the common carotid artery, a similar sheath or sheath/shunt systems may be designed for subclavian access.

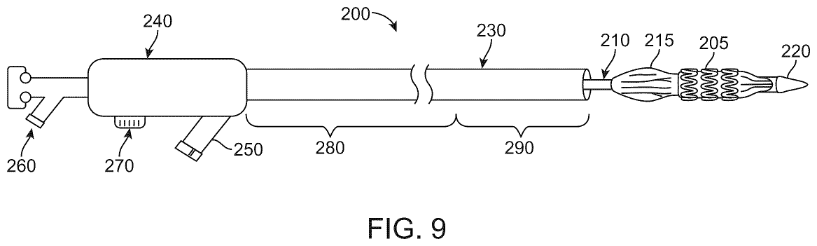

An exemplary valve and delivery system which has been configured to be delivered through the transcarotid access sheath 110 is shown in FIG. 9. The route from the transcarotid access site is fairly short and straight, as compared to the transfemoral or subclavian approach. As a result, the delivery system can be shorter and the proximal section can be quite rigid, both of which will allow greater push and torque control resulting in increased accuracy in positioning and deploying the prosthetic valve. The distal section has increased flexibility to allow accurate tracking around the ascending aorta and into position at the aortic annulus. Materials for the delivery system may include reinforced, higher durometer, and/or thicker walled materials as compared to current delivery systems to provide this increased rigidity.

The balloon expandable prosthetic aortic valve 205 is mounted on the distal end of an endovascular valve delivery system 200. The delivery system has a distal tapered tip 220 and an expandable balloon 215 on the distal end of an inner shaft 210. In an embodiment, the system also has an outer sleeve, such as for example a pusher sleeve 230, that is slidable along the long axis of the device and which maintains the valve in position on the balloon during delivery. A proximal control assembly contains a mechanism for retracting the pusher sleeve, such as a sliding button 270 on a proximal handle 240. In FIG. 9, the pusher sleeve 230 is shown retracted from the valve and proximal balloon so that the valve can be expanded without interference from the pusher sleeve 230. A connector 250 allows connection of an inflation device to the balloon inflation lumen of the balloon 215. A proximal rotating hemostasis valve 260 allows system flushing as well as sealing around a guidewire (not shown) as the valve delivery system is being advanced over the guidewire and into position.

The working length of the valve delivery system is configured to allow delivery of the valve to the aortic annulus from a transcarotid access site.

Specifically, the working length of the valve delivery system 200 can be between 45 and 60 cm. The delivery system shaft is also configured for delivery from a right or left carotid access site. Specifically, the shaft has a proximal stiff section 280 and a more flexible distal section 290. In an embodiment, the distal section is 2 to 4 times more flexible than the proximal stiff section. In an embodiment, the distal flexible section is between one quarter to one third the total working length of the valve delivery system. Specifically, the distal flexible section is in the range 10 cm to 20 cm. In an alternate embodiment, the valve delivery system has a transition section of one or more flexible lengths which fall between the flexibility of the distal flexible section and the proximal flexible section.

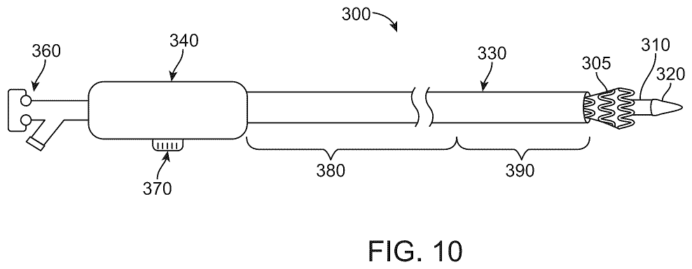

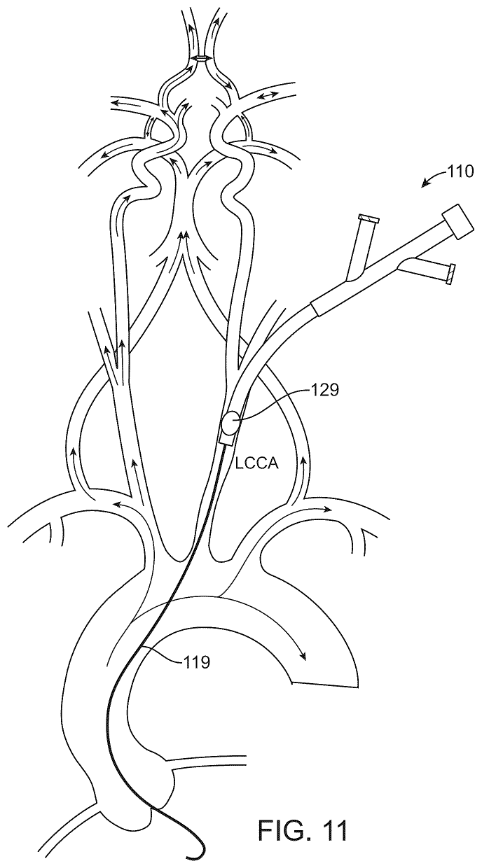

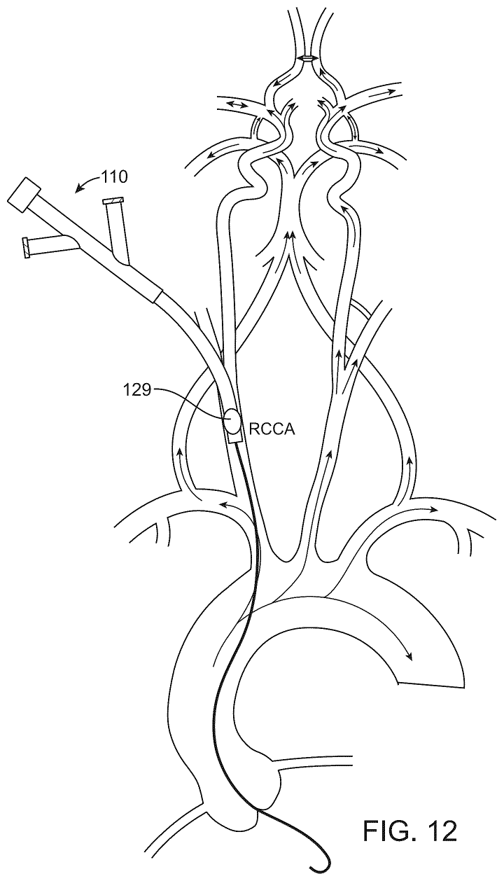

Another exemplary valve and delivery system configured for transcarotid delivery is shown in FIG. 10. The self-expanding prosthetic aortic valve 305 is mounted on the distal end of an endovascular valve delivery system 300. The delivery system has a distal tapered tip 320 on the distal end of an inner shaft 310. The valve 305 is positioned on the inner shaft 310 and contained in a retractable sleeve 330 that can slide along the longitudinal axis of the device. A proximal control assembly contains a mechanism for retracting the retractable sleeve, such as a sliding button 370. In an embodiment, the design of the valve 305 and sleeve 330 are such that the sleeve can be readvanced in a distal direction to abut and to collapse the valve so that the valve 305 can be re-positioned if the first position was inaccurate. A proximal rotating hemostasis valve 360 allows system flushing as well as sealing around a guidewire (not shown) as the valve delivery system is being advanced over the guidewire and into position.

As with the previous embodiment, the working length of the valve delivery system is configured to allow delivery of the valve to the aortic annulus from a transcarotid access site. Specifically, the working length of the valve delivery system 300 is between 45 and 60 cm. The delivery system shaft is also configured for delivery from a right or left carotid access site. Specifically, the shaft has a proximal stiff section 380 and a more flexible distal section 390. In an embodiment, the distal section is 2 to 4 times more flexible than the proximal stiff section. In an embodiment, the distal flexible section is between one quarter to one third the total working length of the valve delivery system. Specifically, the distal flexible section is in the range 10 cm to 20 cm. In an alternate embodiment, the valve delivery system has a transition section of one or more flexible lengths which fall between the flexibility of the distal flexible section and the proximal flexible section.

Exemplary methods of use are now described. In an embodiment, a general method includes the steps of forming a penetration from the neck of a patient into a wall of a common carotid artery (or other access location such that this disclosure is not limited to entry at the common carotid artery); introducing an access sheath through the penetration with the tip directed inferiorly towards the ostium of the artery; inserting a guide wire through the access sheath into the ascending aorta and across the native aortic valve; and introducing a prosthetic valve through the access sheath and percutaneously deploying the prosthetic valve at or near the position of the native aortic valve. In an embodiment, the artery is occluded distal (upstream) from the tip of the sheath.

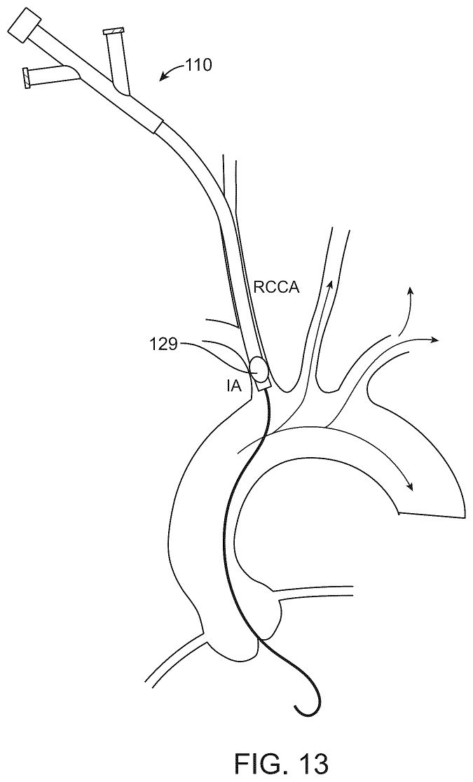

In particular, the access sheath 110 is first inserted into the vasculature such as via either a percutaneous puncture or direct surgical cut-down and puncture of the carotid artery. As mentioned, a transcarotid approach to the aortic valve may be achieved via the LCCA. Once properly positioned, the occlusion element 129 may be expanded to occlude the LCCA, as schematically shown in FIG. 11. In another embodiment, a transcarotid approach to the aortic valve may be achieved via the RCCA, with the occlusion element 129 occluding the RCCA, as shown in FIG. 12. In another embodiment, a transcarotid approach to the aortic valve may be achieved via the RCCA, with the occlusion element 129 occluding the innominate artery IA, as shown in FIG. 13. The occlusion achieved via the occlusion element 129 can also be achieved via direct clamping of the carotid vessel, e.g. with a vascular clamp, vessel loop or Rummel tourniquet.

Once the access sheath is positioned and the embolic protection means are deployed via occlusion, aspiration, and/or filter elements, access to the aortic valve is obtained via a guidewire 119 (such as a 0.035″ or 0.038″ guidewire) inserted into the sheath 110 and directed inferiorly into the ascending aorta and across the native aortic valve. Pre-dilation of the native aortic valve can be performed with an appropriately sized dilation balloon, for example a valvuloplasty balloon, before valve implantation. The guidewire 119 is used to position a balloon across the valve and the balloon is inflated, deflated, and then removed while the guidewire remains in place.

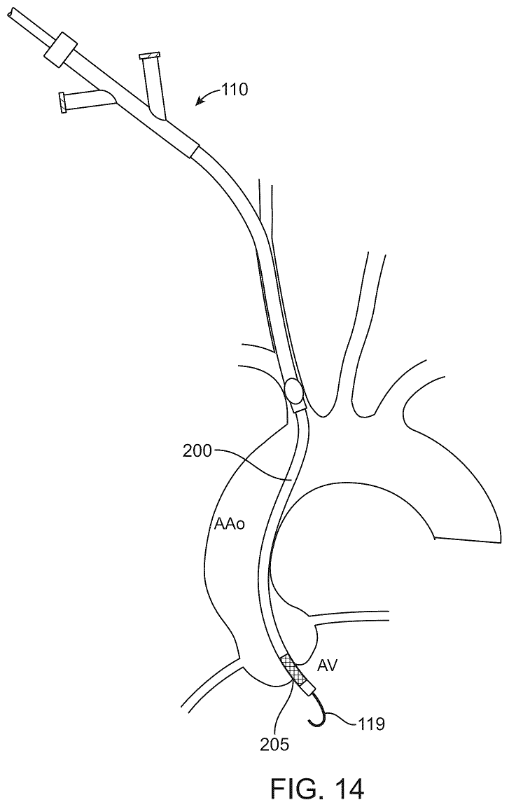

An endovascular prosthetic valve 205 and delivery system 200 (or delivery system 300) is then inserted through the access sheath 110 over the guidewire 119 and the valve 205 positioned at the site of the native aortic valve (as shown in FIG. 14). The prosthetic valve 205 is then implanted. At the conclusion of the implantation step, the implanted prosthetic valve 205 function can be accessed via ultrasound, contrast injection under fluoroscopy, or other imaging means. Depending on the design of the delivery system 200, the prosthetic valve 205 may be adjusted as needed to achieve optimal valve function and position before final deployment. The delivery system 200 and guidewire 119 are then removed from the access sheath 110. After removal of the delivery system 200 and guidewire 119, the embolic protection elements are removed. Aspiration may continue during this time to capture any embolic debris caught in the sheath tip, occlusion element and/or filter elements.

The access sheath 110 is then removed and the access site is closed. If the access was a surgical cutdown direct puncture, the vessel is closed either via tying off the pre-placed stitch or with manual suturing or with a surgical vascular closure device, as described in more detail below. If the access was percutaneous, percutaneous closure methods and devices may be employed to achieve hemostasis at the access site. In an embodiment, the closure device is applied at the site of the penetration before introducing the arterial access sheath through the penetration. The type of closure device can vary.

The access site described above is either the left or right common carotid artery. Other access sites are also possible, for example the left or right subclavian artery or left or right brachial artery. These arteries may require longer and/or more tortuous pathways to the aortic valve but may offer other advantages over a carotid artery access, for example the ability to work away from the patient's head, the ability to avoid hostile neck anatomy such as previous carotid endarterectomy or other cervical surgery or radiation, or less risky in case of access site complication. In addition, carotid artery disease, or small carotid arteries may preclude common carotid artery access. In the case of any of these access sites, occlusion, aspiration, and/or filtering the head and neck vessels during TAVI may increase the speed and accuracy of the procedure, and decrease the rate of embolic complications.

If the access to the carotid artery was via a surgical cut down, the access site may be closed using standard vascular surgical techniques. Purse string sutures may be applied prior to sheath insertion, and then used to tie off the access site after sheath removal. If the access site was a percutaneous access, a wide variety of vessel closure elements may be utilized. In an embodiment, the vessel closure element is a mechanical element which include an anchor portion and a closing portion such as a self-closing portion. The anchor portion may comprise hooks, pins, staples, clips, tine, suture, or the like, which are engaged in the exterior surface of the common carotid artery about the penetration to immobilize the self-closing element when the penetration is fully open. The self-closing element may also include a spring-like or other self-closing portion which, upon removal of the sheath, will close the anchor portion in order to draw the tissue in the arterial wall together to provide closure. Usually, the closure will be sufficient so that no further measures need be taken to close or seal the penetration. Optionally, however, it may be desirable to provide for supplemental sealing of the self-closing element after the sheath is withdrawn. For example, the self-closing element and/or the tissue tract in the region of the element can be treated with hemostatic materials, such as bioabsorbable polymers, collagen plugs, glues, sealants, clotting factors, or other clot-promoting agents. Alternatively, the tissue or self-closing element could be sealed using other sealing protocols, such as electrocautery, suturing, clipping, stapling, or the like. In another method, the self-closing element will be a self-sealing membrane or gasket material which is attached to the outer wall of the vessel with clips, glue, bands, or other means. The self-sealing membrane may have an inner opening such as a slit or cross cut, which would be normally closed against blood pressure. Any of these self-closing elements could be designed to be placed in an open surgical procedure, or deployed percutaneously.

In an alternate embodiment, the vessel closure element is a suture-based vessel closure device. The suture-based vessel closure device can place one or more sutures across a vessel access site such that, when the suture ends are tied off after sheath removal, the stitch or stitches provide hemostasis to the access site. The sutures can be applied either prior to insertion of a procedural sheath through the arteriotomy or after removal of the sheath from the arteriotomy. The device can maintain temporary hemostasis of the arteriotomy after placement of sutures but before and during placement of a procedural sheath and can also maintain temporary hemostasis after withdrawal of the procedural sheath but before tying off the suture. U.S. patent application Ser. No. 12/834,869 entitled “SYSTEMS AND METHODS FOR TREATING A CAROTID ARTERY”, which is incorporated herein by reference in its entirety, describes exemplary closure devices and also describes various other devices, systems, and methods that are related to and that may be combined with the devices, systems, and methods disclosed herein.

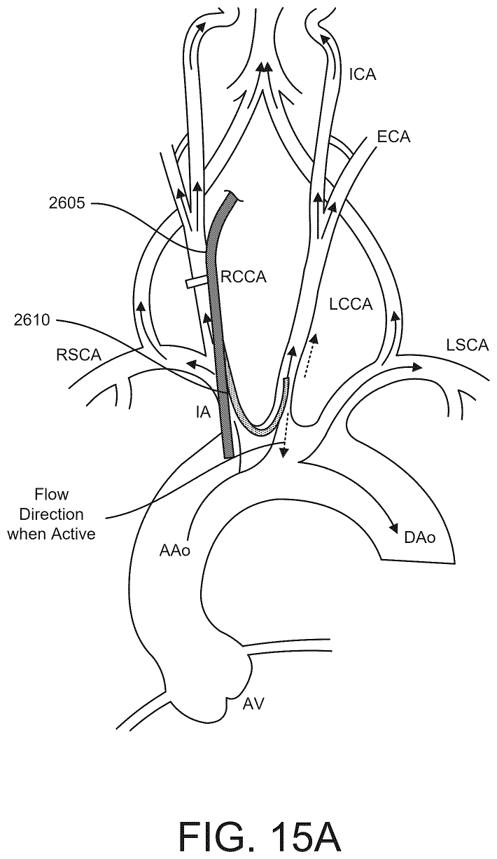

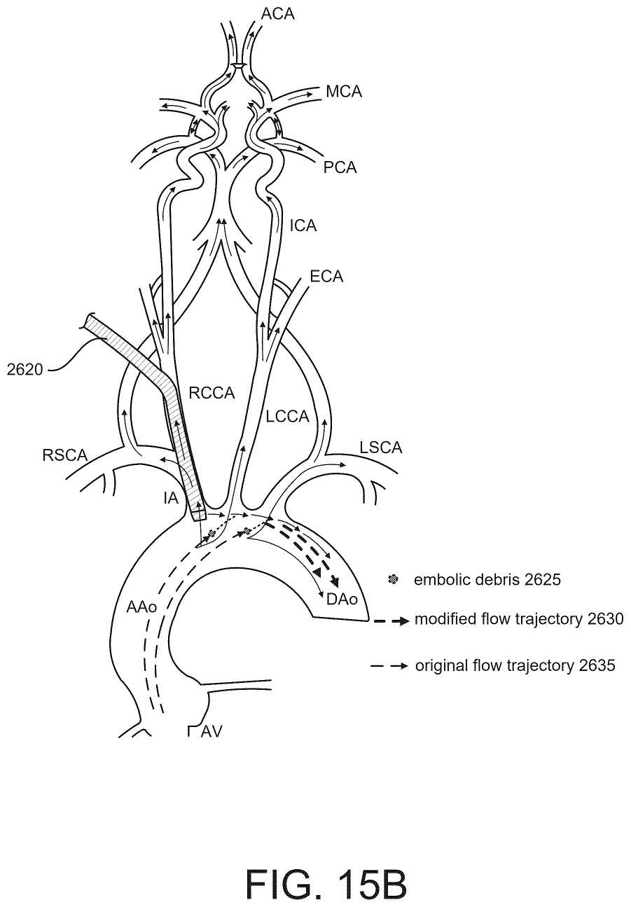

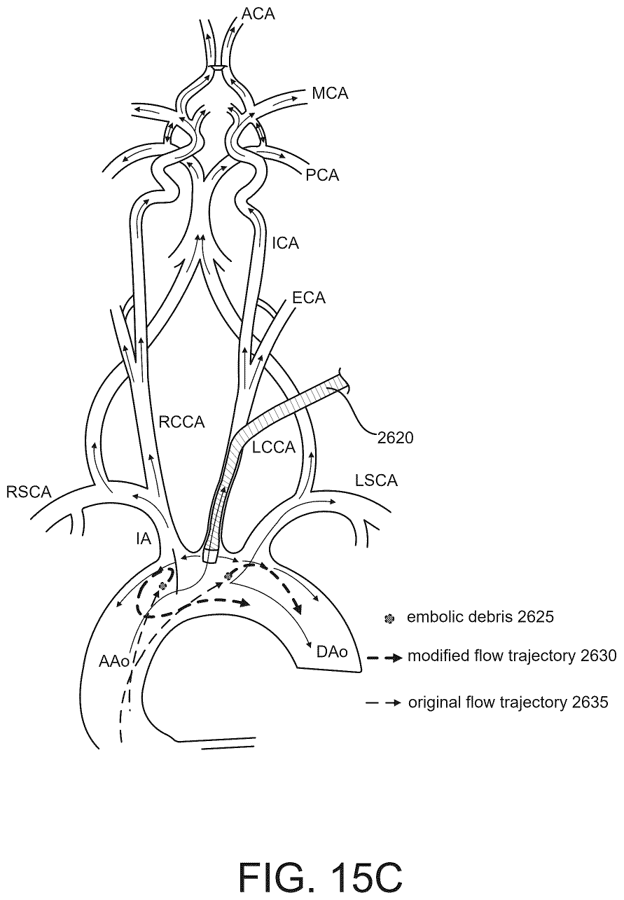

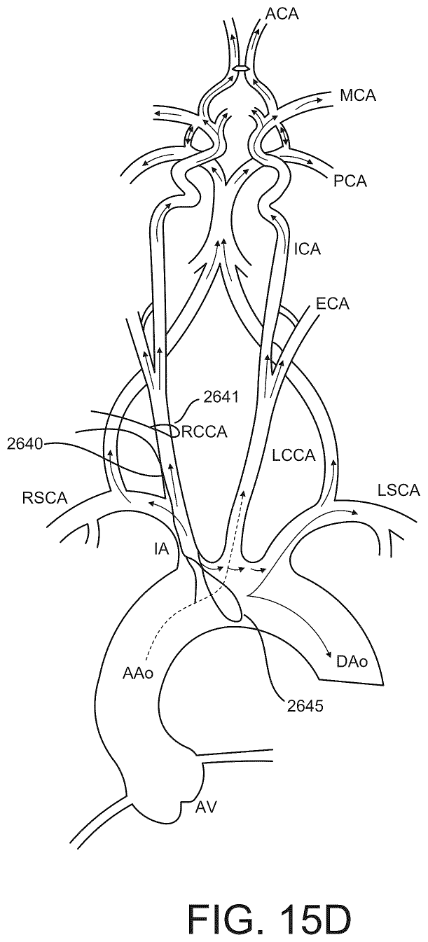

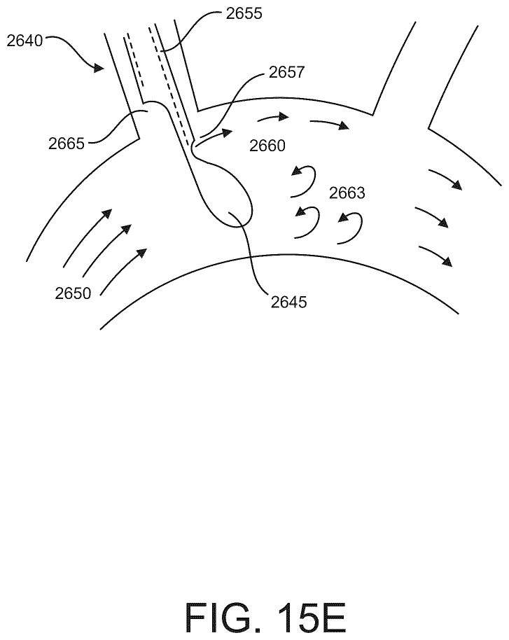

FIG. 15A shows a schematic representation of an embodiment wherein an access sheath 2605 is used to gain transcervical access to the RCCA. A flow catheter 2610 also accesses the LCCA via insertion through the access sheath 2605. The RCCA is clamped (or otherwise occluded) to stop or otherwise partially stop blood flow through the RCCA. The flow catheter 2610 can be used to inject fluid (such as saline for example) into the contralateral common carotid artery (or other location) such as during instances where an interventional procedure may generate embolic particles. The injected fluid is configured to divert those particles away from the neurovasculature such as away from predetermined locations of the neurovasculature. The injected fluid may be referred to as a “fluid curtain” wherein the fluid curtain or injected fluid achieves flow conditions, such as a flow velocity or direction of flow that redirects embolic debris along a modified flow trajectory that is different from an original flow trajectory that would occur absent the presence of the fluid curtain.

In this embodiment, there may be two, non-limiting example modes of operation:

Sub-systolic pressure: A predetermined pressure, such as a pressure of approximately 100 mmHg, is maintained which will only flow forward and divert flow and embolic particles during times that patient blood pressure drops significantly. The blood pressure drops such as during implantation of an aortic valve when temporary pacing is conducted to stabilize the aortic valve. This step of the procedure may generate significant embolic particles, and the flow diversion is naturally triggered by the decrease in patient blood pressure.

Supra-systolic pressure: Flow diversion is activated via the injected fluid at pressures slightly higher than systolic blood pressure during discrete time periods of the procedure to block incoming blood flow to minimize embolic particles from passing from the aortic valve to the carotid artery.

Example procedural steps pursuant to the embodiment of FIG. 15A include: First, access to the desired CCA is achieved and an access system (such as the access sheath 2605 or other conduit) is used to gain access to the vasculature. Next, the CCA that was accessed is occluded to stop or otherwise limit flow therethrough. Occlusion can occur for example via a clamp or an expandable element such as a balloon sheath. The flow catheter 2610 is then delivered to the contralateral CCA (through or adjacent to the access sheath 2605). Saline (or other fluid) is then injected to CCA as desired per mechanisms described herein. A cardiac or aortic procedure is performed as intended through access system. Flow reversal may optionally be achieved in the access side CCA to increase neuroperfusion.

FIG. 15B shows a schematic view of the vasculature with a catheter 2620 positioned to access the aortic arch (which defines an aortic annulus) via the RCCA such that the catheter 2620 can inject fluid into the aortic arch or other desired location. In an alternate version, the catheter 2620 may be an access sheath having at least one internal lumen and at least one outlet for injecting fluid in the same or similar manner as described for the catheter 2620. The catheter 2620 has at least one fluid outlet that directs a flow of fluid out of the catheter such that embolic debris 2625 follows a modified flow trajectory 2630 that is different from an original trajectory. The redirected particles can be solid particles and/or air particles. The catheter 2620 can have one or more outlets configured to achieve a desired flow profile for fluid that is directed out of the outlet(s).

FIG. 15C shows a schematic view of the vasculature with a catheter 2620 positioned via the LCCA so as to be able to inject fluid into the aortic arch such as via a lumen of the catheter 2620 wherein fluid exits an opening in a distal region or distal end of the catheter 2620. In this embodiment, the catheter 2620 has two or more outlets to achieve at least two flows of fluid that direct fluid flow in at least two different directions. The injected fluid causes embolic debris 2625 to follow at least one modified flow trajectory 2630 that is different from an original flow trajectory 2635.

FIG. 15D shows a schematic view of the vasculature with a catheter (or sheath) 2640 inserted into the RCCA to access the aortic arch. The RCCA is clamped (such as via a Rummel loop 2641) adjacent the catheter 2640. The catheter 2640 is configured to inject fluid into the vasculature such as into the aortic arch. The catheter 2640 is attached to (or can be used to deliver) an aerodynamic or hydrodynamic deflector element such as a spoiler 2645, which is positioned at or near a distal end of the catheter 2640. The catheter can be used to position the spoiler 2645 in the aortic arch such that spoiler disrupts or otherwise adjusts fluid flow through the aortic arch in a desired manner. The spoiler has at least one outer surface that is configured to interact with the fluid flow in a manner that adjusts or modifies the fluid flow. In an embodiment, the spoiler 2645 creates a flow bias of blood flow coming from aortic valve to be deflected away from the CCA or to be deflect away from a predetermined location in the vasculature. A flow curtain occurs behind (such as closer to the CCA) such as to minimize deflected particles from swirling or otherwise flowing into the CCA. The flow curtain can be created by a hydrodynamic spoiler effect or the flow curtain can be used in combination with a spoiler that does not cause the flow curtain. The spoiler 2645 can be made of various materials. In an example, the spoiler 2645 is made of Ultra-high-molecular-weight polyethylene (UHMWPE) supported by a wire frame.

FIG. 15E shows a schematic view (not to scale) of a distal region of the catheter 2640 positioned in the aortic arch via the RCCA. The spoiler 2645 is positioned within the aortic arch downstream of laminar aortic blood flow 2650. The catheter 2640 includes a proximal large bore region that transitions into a smaller diameter region having an internal lumen 2655 that can be fluidly coupled to a source of fluid (such as blood shunted from another vascular location.) An outlet 2657 of the internal lumen 2655 provides a pathway for fluid (such as shunted fluid) to be injected into the aortic arch. The fluid injected via the outlet 2657 can initially be laminar flow 2660. In addition, the spoiler 2645 disrupts or otherwise modifies the laminar aortic blood flow 2650 to produce a region of turbulent aortic blood flow 2663.

With reference still to FIG. 15E, the catheter 2640 also includes an opening 2665 that communicates with a larger internal lumen of the catheter 2640. The larger internal lumen and opening 2665 can be used for delivery of an interventional device such as a valve delivery device. It should be appreciated that the embodiment shown in FIGS. 15D and 15E can be modified for approach via the LCCA. The lumen(s) of the device and the relative positions of the spoiler can be accordingly modified.

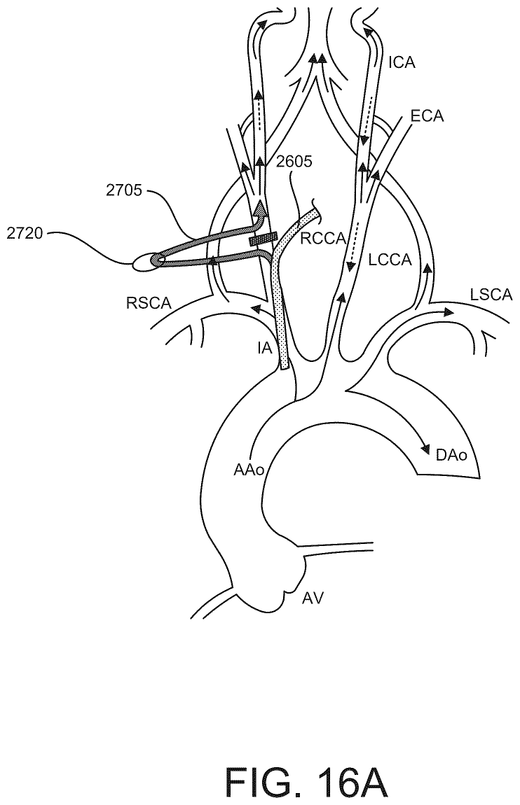

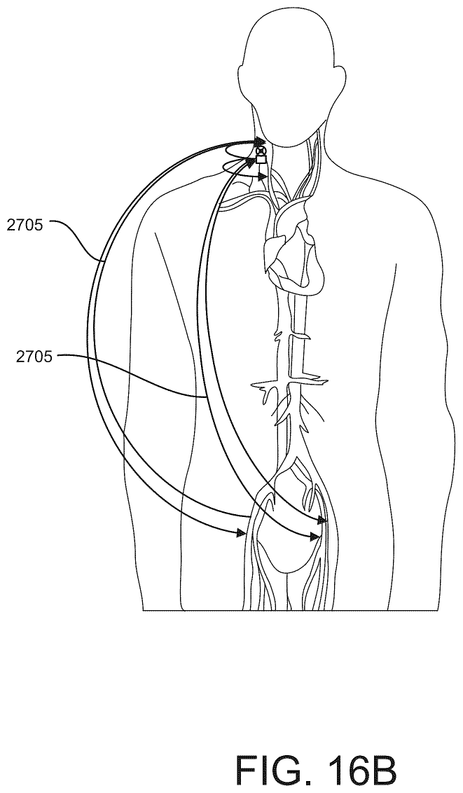

FIG. 16A shows a schematic representation of another embodiment wherein an arterial shunt 2705 is combined with or otherwise coupled to the access sheath 2605. The shunt 2705 has an internal lumen such that blood flow can be routed from a first location to a second location via the shunt 2705. Pursuant to an example procedure, access to the desired CCA (left or right) is achieved and an access system (such as the access sheath 2605 or other conduit) is used to gain access to the vasculature. Next, the CCA that was accessed is occluded such as by using a clamp or an expandable balloon to form a carotid stump region above the clamp (or balloon). The carotid stump region above the location of the clamp is also accessed using the arterial shunt 2705. As mentioned, the arterial shunt 2705 has an internal lumen that forms an antegrade flow pathway (a bypass or shunt) between the two points of access across the location of the clamp. A cardiac or aortic procedure is then performed via the access sheath 2605.

With reference still to FIG. 16A, the shunt 2705 may include one or more blood flow control elements 2720 configured to modify or adjust the state of blood flow through the shunt 2705. For example, the flow control element 2720 can include a filter to capture debris such as embolic particles of determined size. The flow control element 2720 can also include an air-trap or bubble trap to capture air. This addresses the risk of air being introduced to the bloodstream during a procedure via the shunt. The flow control element 2720 can also be configured to adjust an aspect of blood flow such as to achieve desired flow conditions and/or to meet patient tolerances. The size of the filter (or any filter discussed herein) may vary. In a non-limiting example, the filter is a 200 micrometer pore size filter or a smaller filter such as 50 um or 100 um pore size filter.

The flow through the shunt may occur passively such that blood flow is driven by naturally occurring pressure differentials. For example, there may be a higher pressure from the RCCA below the carotid clamp and a lower pressure from the RCCA above the carotid clamp. The flow may also occur actively such that flow is driven by a pump mechanism. In a non-limiting example, the blood is passively or actively pushed across the Circle of Willis (CoW) to provide neuroperfusion during a treatment procedure. Forward flow across the CoW may deter particles from traveling up the contralateral side, as represented by the dashed arrow elements in FIG. 16A.

This procedure allows blood to be manipulated to safely achieve neuroprotective benefits such as perfusion of blood to the brain, capture of debris from the blood stream during the procedure, and pushing of antegrade blood flow through or to the brain such as to deter debris from flowing up the contralateral vessels.

The arterial shunt 2705 can have any of a variety of lengths and sizes to achieve desired flow characteristics and to achieve connection between different locations of the vasculature. The length may be shorter such as to shunt flow across more local vessels (such as a carotid-carotid or carotid-jugular pathway) or longer for shunting across more distant locations (such as carotid-iliac pathway.) FIG. 16B shows a schematic representation of possible flow routes that can be achieved by use of the shunt 2705. FIG. 16B shows this in in the context of a right-side carotid access although left-side access is within the scope of this disclosure.

The shunt 2705 can be connected to the blood stream in various ways such as via the sheath 2605 (as shown in FIG. 16A) or via a secure connection to a compatible device or through a surgically attached conduit.

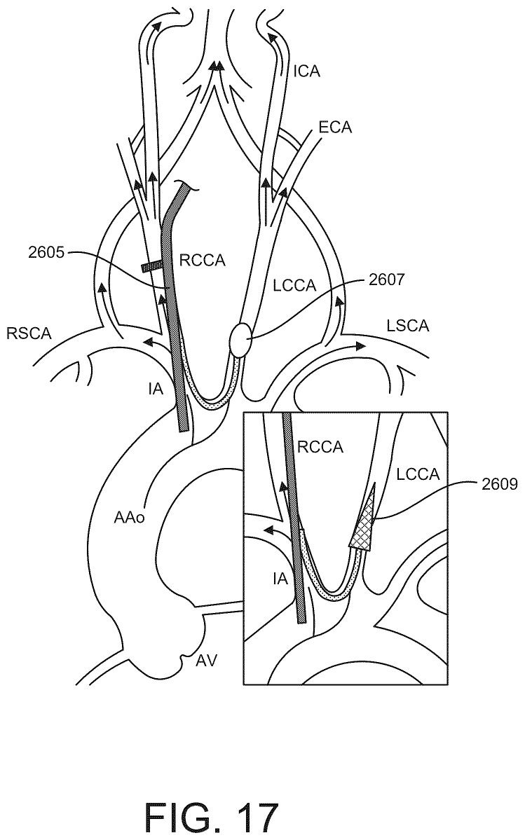

FIG. 17 shows an embodiment wherein a balloon 2607 is placed in the contralateral CCA to provide additional cerebral protection by occluding flow (or partially occluding flow) in the contralateral CCA. Partial occlusion may also be sufficient to reduce flow and reduce embolic particles. In an alternate embodiment, an embolic filter may be positioned in the contralateral CCA (or other location) to provide additional cerebral protection without occluding flow. Pursuant to an example procedure, access to the desired CCA is achieved and an access system (such as the access sheath 2605 or other conduit) is used to gain access to the vasculature. Next, the CCA that was accessed is occluded. A balloon catheter or embolic filter 2609 is deployed to the contralateral CCA via the access system. A cardiac or aortic procedure is performed as intended through access system.

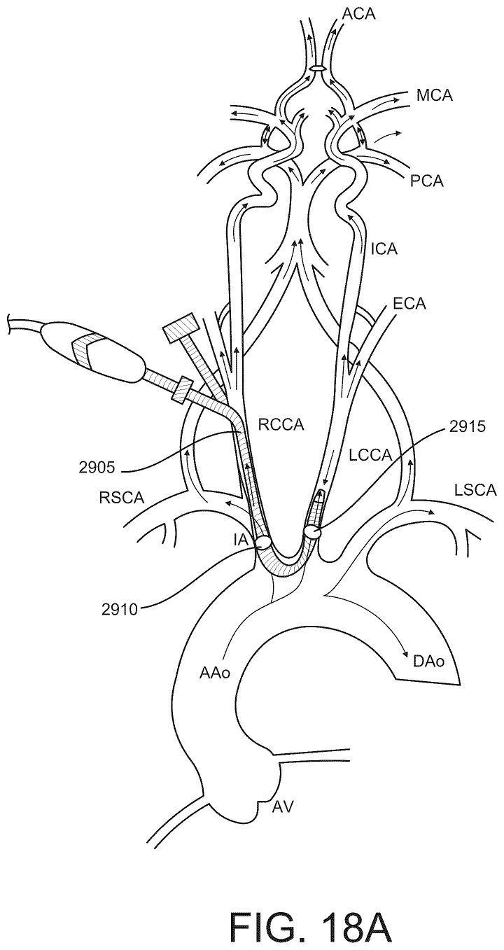

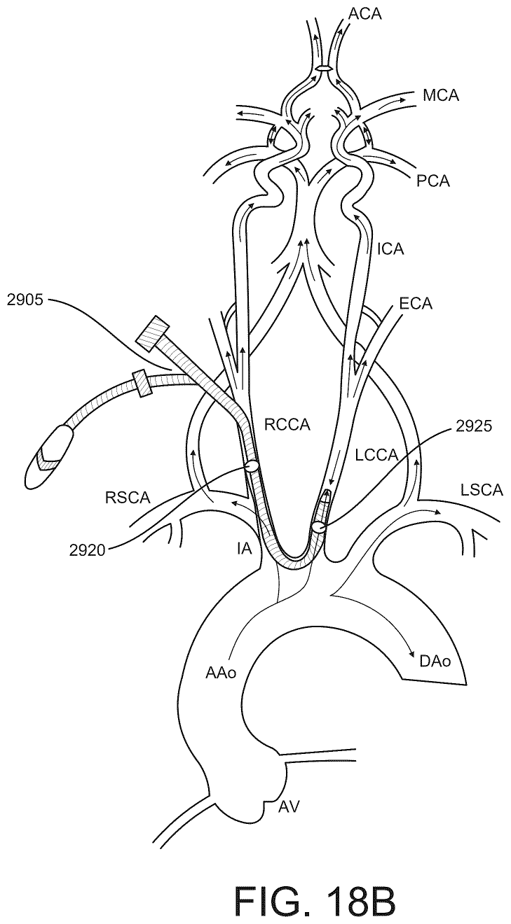

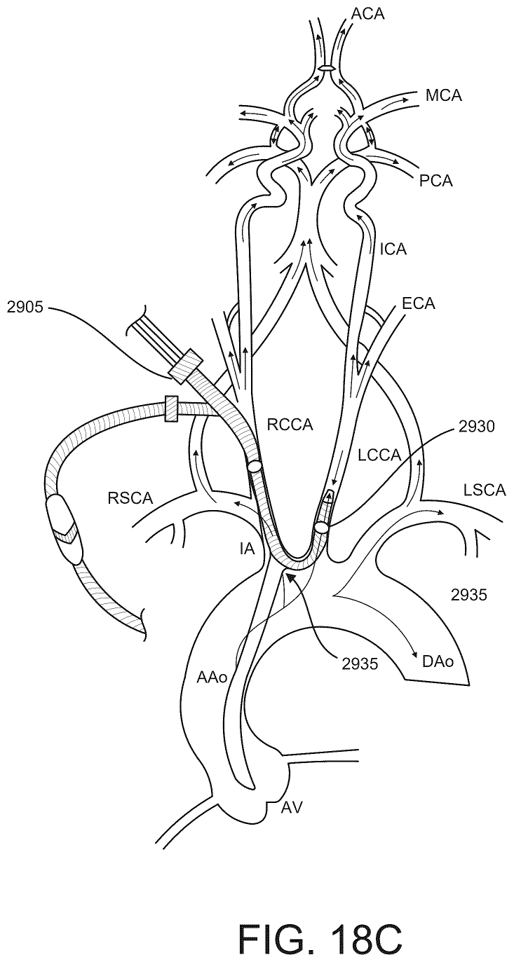

FIGS. 18A through 18C show additional embodiments wherein one or more occlusion balloon catheters are used. These may be used, for example, to achieve embolic protection of the great blood vessels during large bore procedures. The use of an occlusion balloon may achieve occlusion of flow and a reverse flow mechanism to provide both embolic protection and brain perfusion. Any catheters that are used may be steerable to permit or enhance guidance to neighboring vessels. These and any of the disclosed configurations are tailorable to accessing from both the right carotid artery and left carotid artery. Various features can be tailored to accommodate multiple anatomy configurations such as the number, size and location of the balloons, as well as the location of any openings for device access. The balloons may also be sized to cover a wider diameter range to enable occlusion for both carotids and innominate arteries, which can provide for a more a versatile and adaptable device.

FIG. 18A shows an embodiment wherein a balloon catheter 2905 is deployed into the RCCA. The balloon catheter 2905 has a first balloon 2910 that is positionable in the innominate artery and a second balloon 2915 that is positionable in the LCCA. The balloons may be expanded to achieve partial or complete flow occlusion through the respective vessel.

FIG. 18B shows another embodiment wherein a balloon catheter 2905 is deployed into the RCCA and that further extends into the LCCA via the aortic arch. The balloon catheter 2905 has a first balloon 2920 that is positionable in the RCCA and a second balloon 2925 that is positionable in the LCCA.

FIG. 18C shows an embodiment wherein a balloon catheter 2905 is deployed into the RCCA. The balloon catheter 2905 has a balloon 2930 that is positionable in the LCCA. The catheter 2905 also has an opening 2935 along its length that can be positioned to provide access to the aorta via the catheter 2905. The opening 2935 can be used to insert an interventional device such as to permit aortic valve delivery. The size of the opening 2935 can vary to provide access therethrough of different sized devices.

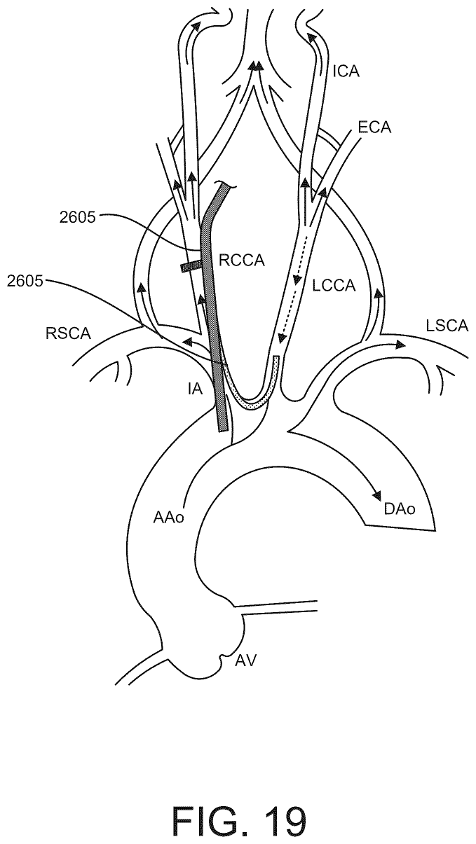

FIG. 19 shows an embodiment wherein blood is pulled through an aspiration catheter placed in the contralateral CCA to improve perfusion and divert particles away from the neurovasculature. Blood can be pulled through a shunt originating in the contralateral CCA to improve perfusion and divert particles away from the neurovasculature. First, access to the desired CCA is achieved and an access system (such as the access sheath 2605 or other conduit) is used to gain access to the vasculature. Next, the CCA that was accessed is occluded. A flow catheter is delivered to the contralateral CCA such as through or adjacent the access system. Aspiration or reverse flow via a shunt is then engaged. A cardiac or aortic procedure is performed as intended through access system. Flow reversal may optionally be achieved in the access side CCA to increase neuroperfusion.

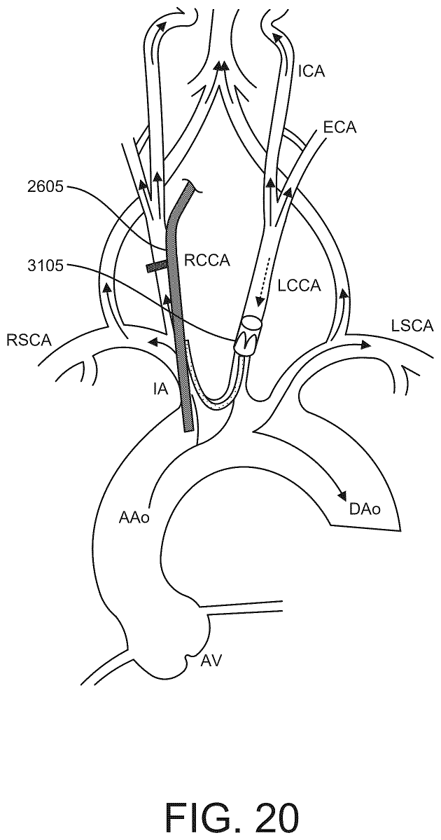

FIG. 20 shows an embodiment wherein a vascular valve 3105 is placed in the contralateral CCA to prevent debris from flowing to the brain. The valve may be configured to allow some pulsatile backflow to support perfusion. The valve may be combined with an aspiration procedure or shunt mechanism to evacuate any trapped debris from the valve and further promote perfusion. The orientation of the valve may be reversed to act as pressure sensitive check-valve so as to allow forward (superior) blood flow at times of high pressure and to block flow during times of low pressure (e.g. during pacing). First, access to the desired CCA is achieved and an access system (such as the access sheath 2605 or other conduit) is used to gain access to the vasculature. Next, the CCA that was accessed is occluded. The valve is delivered to the contralateral CCA. A cardiac or aortic procedure is performed as intended through access system. Flow reversal may optionally be achieved in the access side CCA to prevent or reduce accumulation of valve debris and to increase neuroperfusion.

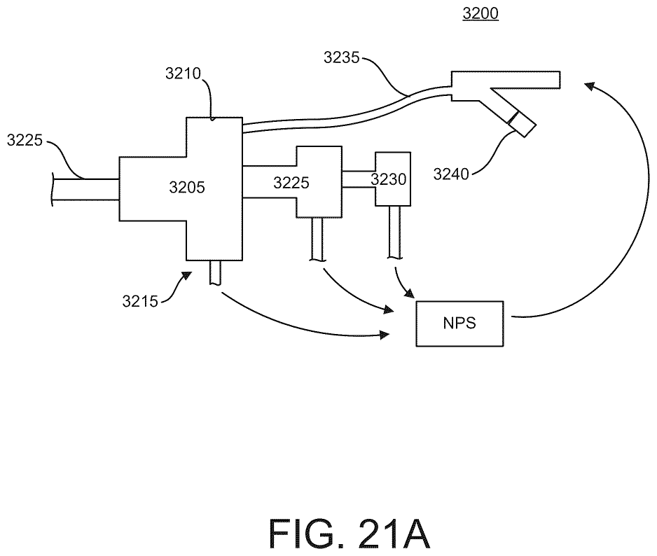

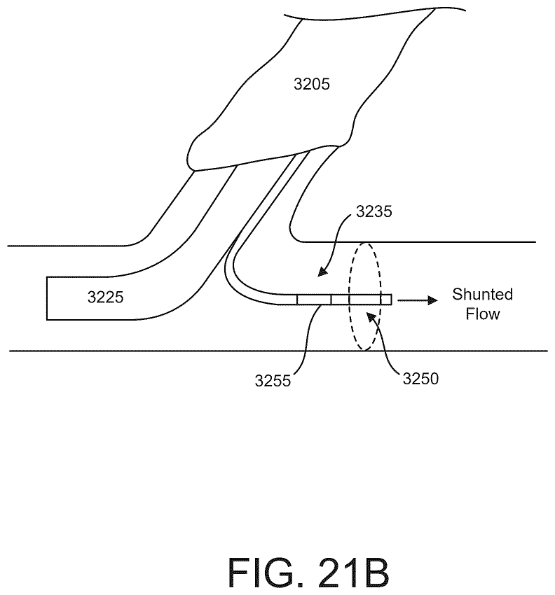

FIG. 21A shows a schematic representation of an embodiment of a conduit and shunt system 3200. The system 3200 includes a conduit 3205 that can be sewn or otherwise attached to a blood vessel such as the CCA. When attached as such, the conduit 3205 forms a pathway between the internal lumen of the CCA and an internal lumen of the conduit 3205. U.S. patent application Ser. No. 17/555,127 entitled “Vascular conduit to facilitate temporary direct access of a vessel” describes an example conduit and is incorporated herein by reference in its entirety. The conduit 3205 has an integrated hub 3210 that communicates with the internal lumen of the CCA. The hub 3210 may include a hemostasis valve to provide access for one or more devices to the CCA via the conduit 3205. The conduit 3205 can further includes at least one side port 3215 for flushing and/or flow shunting from one location to another via the hub 3210. The side port 3215 can be used to shunt blood flow through the conduit to a neuroprotection system (NPS) that includes a blood filter and/or an air trap. The neuroprotection system can be for example a housing that contains a filter and/or an air trap and through which the shunted blood flows. The integrated hub of the conduit provides access to the blood vessel retrograde in the direction of blood flow.

In the embodiment of FIG. 21A, the system 3200 includes one or more sheaths 3225 and 3230 that also access the blood vessel such as by being inserted through a lumen of the conduit 3205. The sheaths can be configured to support an interventional procedure such as a TAVR procedure or other procedure. Each of the sheaths can have a respective side port for flushing and/or flow shunting. The side ports can also direct blood through the NPS.

The system 3200 may also include a secondary shunting catheter 3235 that can also be used to access the blood vessel in the antegrade flow manner. The secondary shunting catheter 3235 has an inner lumen that enables shunting of blood flow via the conduit hub 3210 after the blood has passed through the NPS. The arrows in FIG. 21A represent blood flow to the secondary shunting catheter 3235. In this manner, the shunted blood flow is free of emboli larger than a certain size, such as larger than 40-100 microns for example. The NPS also removes air bubbles from the shunted blood.

FIG. 21B shows a schematic representation of a distal region of the conduit 3205 with a distal region of the secondary catheter 3205 providing a pathway extending into the blood vessel. The secondary shunting catheter 3235 can have an expandable balloon 3250 positioned at or near a distal tip of the secondary shunting catheter 3235 that can be inflated via an inflation lumen and port 3240 (FIG. 21A) on the proximal region of the secondary shunting catheter 3235. The balloon 3250 is shown in phantom in an expanded state (not to scale.) The balloon can be positioned in the vessel and expanded to stop antegrade blood flow and redirect such flow through a pathway that includes the conduit 3205, the NPS, the secondary shunting catheter 3235 and to a return location. The secondary shunting catheter 3235 can also have a predetermined shape, such as a curved or steerable distal region configured to direct the secondary shunting catheter 3235 in an antegrade direction when the secondary shunting catheter enters the vessel via the conduit 3205.

In an embodiment, the secondary shunting catheter 3235 includes a region 3255 configured to couple with a vascular clamp that is configured to stop antegrade flow through the vessel while sealing around the outer diameter of the secondary shunting catheter but without causing any collapse of the secondary shunting catheter 3235.

In an embodiment, a delivery catheter is deployed through the hemostasis valve of the conduit. The delivery catheter can be used for various activities when the inner lumen of the conduit is not occluded via a device such as an implant delivery device. For example, an inner lumen of the delivery catheter can be used to shunt blood flow in a similar fashion similar the conduit. A sheath may be used in conjunction with the conduit to provide an additional level of embolic protection. It may also increase the volume of blood shunted to an end location such as an organ. It may also be used where a conduit or anastomosis is not used.

The delivery catheter may also have a specialized hub that includes an adapter that permits purging and flushing of the catheter in addition use of the neuroprotection device. In situations where a conduit is not used, the specialized clamp can be used to cease blood flow around the outer diameter of the sheath.

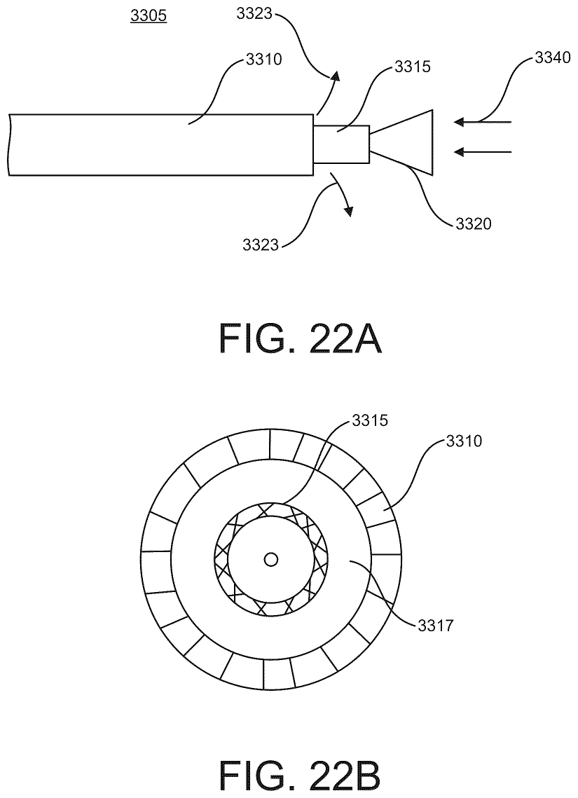

FIG. 22A shows an embodiment of a distal region of a catheter device 3305 configured to capture embolic debris from other devices and/or the patient's vasculature. FIG. 22B shows the device 3305 in cross section. The device 3305 can also be configured to capture air emboli that is released during an intervention such as during an implant delivery. The device 3305 can be positioned into a blood vessel of the vascular such that it captures blood flow through a vascular pathway and routes such blood flow through a neuroprotection system (NPS) that includes a filter and air trap that captures emboli greater than a certain size such as 40-200 microns. The device 3305 advantageously maintains perfusion along the pathway by returning the blood safely via a co-axial or eccentric pathway of the device 3305 as described below.

With reference to FIGS. 22A and 22B, the device 3305 includes an outer tube 3310 with a co-axial (or eccentric) inner tube 3315 positioned therein. An annular flow return lumen 3317 (FIG. 22B) is positioned between the outer tube 3310 and the inner tube 3315. A funnel member 3320 is positioned within the inner tube 3315 such that the funnel shape of the funnel member 3320 guides fluid into an inner lumen of the inner tube 3315. The funnel member 3320 can initially be constricted in radial size by the inner tube 3315 such as when the funnel member is retracted inside a lumen of the inner tube 3315. The inner tube 3315 can retract away or otherwise move relative to or from the funnel member 3320 so that the funnel member 3320 expands to the shape shown in FIG. 22A. A guidewire can also be deployed via the device. The funnel may be a filter.

Some exemplary and non-limiting specifications for the device 3305 are now cited. The outer tube may have an outer diameter of 0.093 inch and an inner diameter of 0.085 inch. The inner tube may have an inner diameter of 0.057 inch and outer diameter of 0.070 inch. The device 3305 can be sized to be deployed through a guide sheath. As mentioned, these sizes are non-limiting examples. The funnel member 3320 can be for example a polytetrafluoroethylene (ePTFE) covered nitinol funnel.

The device 3305 can be used in various locations including for example the brachiocephalic artery via a right radial artery approach or left subclavian via a left radial approach. The device may also be delivered via the common carotid artery in a non-limiting example.

The device 3305 can be used as follows. The device is delivered into the vasculature via an access location. The inner tube 3315 is retracted to deploy the funnel member 3320 into its funnel shape such as by allowing the funnel member 3320 to expand from a retracted size to an expanded, funnel-shapes size. The funnel member 3320 directs blood flow into the lumen of the inner tube 3315 as represented by the arrows 3340 in FIG. 22. The inner lumen has an outlet that is purged of air and connected to a neuroprotection system such as a neuroprotection filter with a bubble trap. That is, the inner lumen fluidly connects to a shunt or other flow pathway, wherein fluid (such as blood and embolic debris) flows through the neuroprotection filter that captures the debris. The neuroprotection system fluidly connects via a return flow pathway to the annular flow return lumen 3317 (FIG. 22B) of the device 3305. The resultant shunted and filtered blood flow is returned to the patient (as represented by arrows 3323 in FIG. 22A) to a location proximal (such as about 1 cm proximal) of the location where the funnel and distal end of the inner tube are deployed in the vessel. In an embodiment, the inner tube 3315 extends distally past the distal end of the outer tube 3310 by a distance of about 1 cm.

The device 3305 can include or be coupled to an aspiration device, such as a syringe. During the procedure, a user may apply aspiration via the aspiration device to enhance capture of debris and/or clear debris from the filter, such as during a high-embolic-risk portions of the procedure.

Upon completion of the interventional procedure, the shunted blood flow can be discontinued. The funnel member 3320 can then be recaptured into the device 3305. This device and procedure can be used in other locations such as retrograde popliteal artery access to treat iliac and/or femoral artery disease and cannulation/fenestration of thoracic endograft (TEVAR).

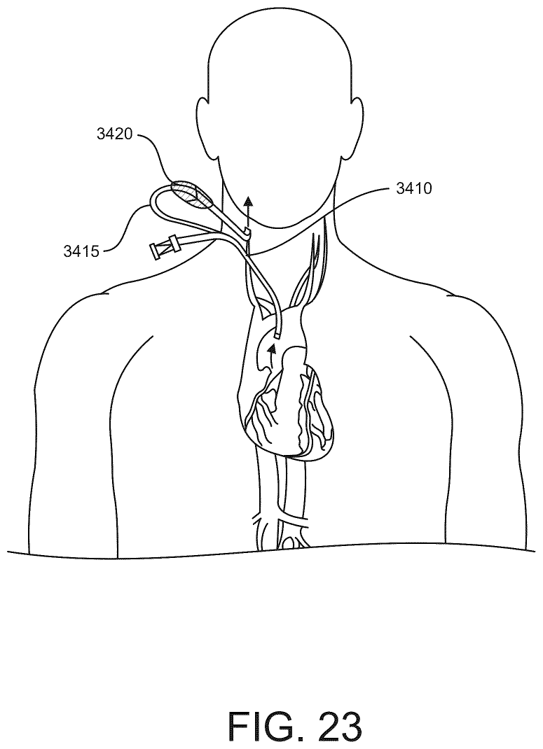

FIG. 23 shows a neuroprotection sheath 3410 deployed in the vasculature such that the sheath includes a distal end or distal region that resides in or otherwise has access to the aortic arch. The sheath 3410 is configured to provide blood perfusion to the brain during a large bore interventional procedure such as a TAVR procedure (or other procedure). The sheath 3410 has a proximal hub that provides hemostatic access to an internal lumen of the sheath 3410. The sheath 3410 includes or is coupled to a shunt 3415 that is configured to shunt a perfusion fluid to the brain such as via the jugular vein where a distal fluid outlet of the shunt 3415 is positioned. The shunt 3415 can have a single outlet or it can have two or more outlets each configured to outlet the filtered, shunted blood in or more directions of the vessel where the outlet(s) are positioned.

Pursuant to this process, blood is shunted from the carotid artery to the ipsilateral jugular vein via the shunt 3415. The shunted blood passed through a filter element 3420 prior to being reintroduced into the patient's body. The shunt 3415 can include a flow control element configured to control a state of blood flow, such as to control blood flow rate between a low and hi rate. The device can be used via an LCCA or an RCCA access location.

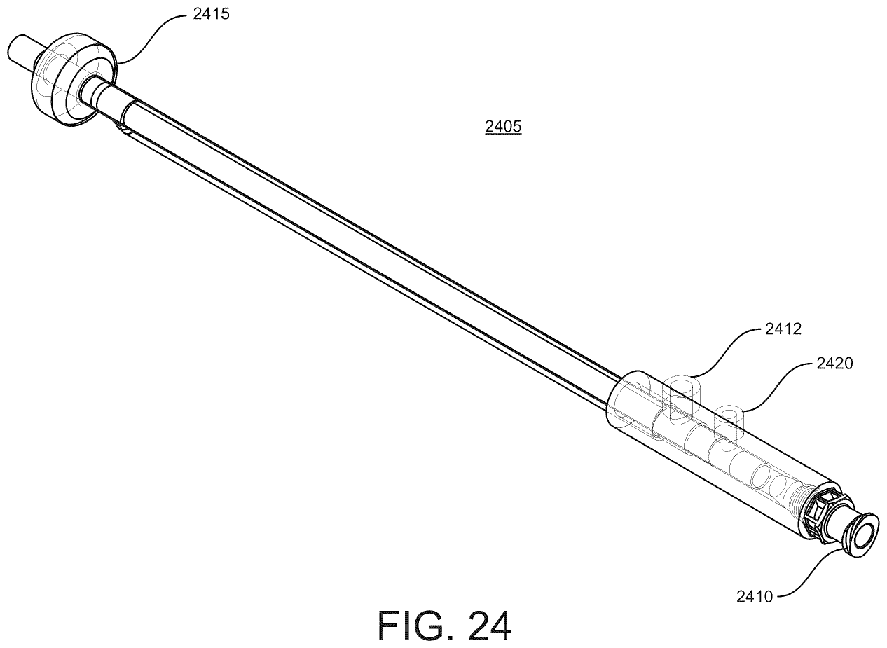

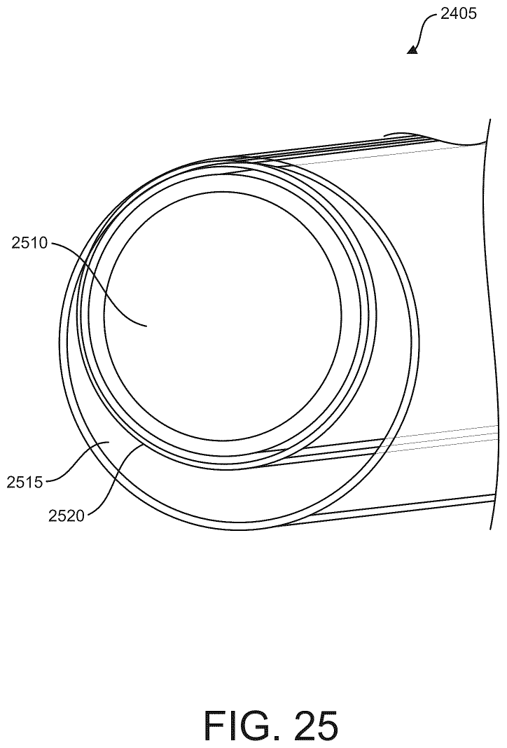

FIG. 24 shows a transparent view of a sheath or cannula 2405 formed of a flexible, elongated body that is sized and shaped to be delivered into the vasculature such as via a percutaneous approach or a surgical incision or cut down approach at an access site of patient. The cannula 2405 is configured for at least four functions including (1) embolic protection via occlusion of a blood vessel (such as the carotid artery, which can alternately be occluded via clamping); (2) capture of embolic material from an artery such as via passive flow of blood into a filter or via active aspiration; (3) reperfusion of a blood vessel such as at a location distal of the access site; and/or (4) delivery of a therapeutic device via an internal lumen of the cannula.

The elongated body has at least one internal lumen including a delivery lumen configured for delivery of a therapeutic device, as well as a lumen for capture of emboli and/or perfusion of an artery. The delivery lumen communicates with an access port 2410 at a proximal region of the cannula 2405 through which a therapeutic delivery device can be inserted. The delivery lumen also communicates with a perfusion port 2412 through which perfusion or shunting of blood to or from the blood vessel can be achieved.

With reference still to FIG. 24, the cannula 2405 includes an expandable element 2415 such as an expandable balloon that can be expanded in an artery to occlude or partially occlude the artery. The expandable element 2415 is located at a distal region of the cannula 2405. The cannula 2405 includes an inflation lumen that communicates with the expandable element 2415 via an inflation hub 2420 at a proximal region of the cannula 2405.

FIG. 25 shows a cross-sectional view of the cannula 2405 and shows a configuration of the lumens. The cannula 2405 includes the delivery lumen 2510, which occupies a center region of the cannula 2405 and which can optionally be co-axial with a long axis of the cannula 2405. The delivery lumen 2510 can vary in size. In an example embodiment, the delivery lumen 2510 is 6-30 French (Fr) in diameter. The delivery lumen 2510 can be used for delivery of therapeutic devices such as guidewires, stents, laser fenestration catheters, needles, or any other device to a blood vessel. The delivery lumen 2510 may also be used to capture emboli via an opening at the distal end of the cannula that communicates with the delivery lumen 2510. In this regard, a therapeutic device can be used to capture the emboli or the emboli may be captured during delivery or deployment of a thoracic endovascular aortic repair (TEVAR) implant. The delivery lumen 2510 may also be used for reperfusion via a deflated balloon. As mentioned, the delivery lumen 2510 communicates with the perfusion port 2412 (FIG. 24), which can be coupled with a Y-adaptor or hemostatic hub that permits passage of devices therethrough and a path for aspiration or flow shunting to a flow controller device having a filter.

With reference still to FIG. 25, the cannula 2405 also includes a shunt lumen 2515 that is offset or non-co-axial with a long axis of the cannula 2405. The shunt lumen 2515 is separated from the delivery lumen 2510 via a wall 2520, which can be an annular wall. A non-co-axial arrangement of the shunt lumen 2515 (relative to a long axis of the cannular 2405) functions to increase or maximize the flow volume within the shunt lumen 2515. The shunt lumen 2515 enables shunting of blood flow to/from the blood vessel and shunting from the delivery lumen 2510 or from a different blood supply such as to maintain neuro perfusion during a procedure.

The cannula 2405 also includes the inflation lumen 2525, which is positioned between the delivery lumen 2510 and the shunt lumen 2515. As mentioned, the inflation lumen 2525 communicates with the inflation port 2420 as shown in FIG. 24.

Pursuant to a method of use, access to the vasculature is achieved via a surgical cut down or a percutaneous access such as at the axillary artery, subclavian artery, carotid artery, or other artery. The cannula 2405 is then inserted into the vasculature to a target location. Insertion can be done with or without a guidewire and/or dilator. The expandable element 2415 is then inflated to occlude and/or seal with the interior arterial wall of a blood vessel at the target location. The dilator and/or guidewire are then removed. The delivery lumen 2510 (FIG. 25) can then be used to capture emboli and blood flow can be shunted to perfuse the artery. These steps can be repeated as needed or desired with one or more additional devices at desired locations. A TEVAR endoprosthesis can be delivered and deployed via the deliver lumen 2510. The artery or arteries can be reperfused as needed.

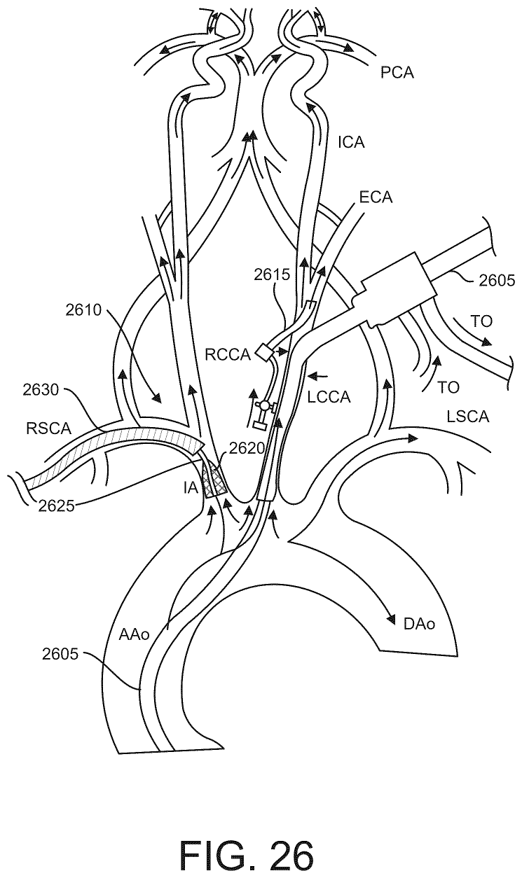

FIG. 26 schematically depicts a view of the vasculature showing normal antegrade circulation. A therapeutic delivery device such as a TAVR delivery device 2605 is deployed via an access location (such as via the LCCA or other location) to the aortic arch via a sheath or cannula device, which can be for example the cannula 2405 described above or other device. The cannula device can be for example a 16-24 French large bore sheath. The RCCA may be occluded (such as via a clamp or via an expandable element inside the artery) at a retrograde direction relative to the access location. A shunt assembly 2615 can be deployed in fluid communication with the cannula device to shunt blood to the LCCA at an antegrade location relative to the access location. The shunt assembly 2615 thus shunts arterial blood flow from an artery and thereby maintain cerebral perfusion.

With reference still to FIG. 26, a filter system 2610 is deployed in the vasculature in communication with the aortic arch. The filter system 2610 is configured to be deployed retrograde to the direction of blood flow such that the filter system 2610 can capture emboli, as described more fully below. The filter system 2610 has at least three mechanisms for emboli capture which improves upon existing embolic protection devices (EPDs) that only use a filter to capture emboli. The filter system 2610 includes a filter 2620 mounted on an inner delivery catheter 2625, which is delivered via an internal lumen of an outer delivery sheath 2630. The sheath 2630 may be for example a 4 F, 5 Fr sheath, 6 Fr sheath, or 7 Fr sheath, which reduces the outer profile compared to existing structural heart embolic protection devices which range in size from 6 F-16 F. The filter may be retracted into the inner lumen of the inner catheter to cause the filter to collapse to a smaller size that fits within the inner lumen. The filter can be funnel shaped with a narrow region of the funnel directly attached to the inner catheter.

The filter 2620 can be a dual layer filter (including an inner filter layer and an outer filter layer) mounted to a frame such as a nitinol frame. The inner filter layer can have a set of pores that differ in size than pores on the outer filter layer. For example, in an embodiment the inner filter layer can have a set of first pores that are smaller than a set of second pores of the outer filter layer such that the inner filter layer has smaller pores or openings relative to the outer layer. The difference in pore size between the inner filter layer and outer filter layer allows materials (such as embolic material) that pass through the inner layer, but not the outer layer, to be trapped between the two layers. In another embodiment, the inner filter layer can have a set of first pores that are larger than a set of second pores of the outer filter layer such that the inner filter layer has larger pores or openings relative to the outer layer. In an alternative embodiment, the filter is only a single layer.

The inner delivery catheter 2625 has an inner diameter (ID) forming an inner lumen such that the inner delivery catheter 2625 can be delivered over a guidewire. The lumen may also be used to capture any material entering the filter 2620 via flow of blood flow through the filter 2620 into the inner lumen. Such material passes into the lumen of the inner delivery catheter 2625. The material then can flow through an extracorporeal neuroprotection system that fluidly communicates with the delivery catheter 2625 and then into an artery or vein. As discussed, the neuroprotection system can include a filter.

In an alternate embodiment, the inner lumen of the inner delivery catheter 2625 has one or more openings or channels that allow fluid communication with an annular space (or outer lumen) between the outer delivery sheath 2630 and the inner delivery catheter 2625. This enables flow to occur through both pathways (i.e., through the lumen of the inner delivery catheter and the annular space) simultaneously. This may be used to increase flow rates, mitigate challenges if one lumen becomes blocked, and assist in preventing formation of clots on the overall system. A guidewire(s) or microcatheter may be partially inserted into the inner lumen of the delivery catheter 2625 to dislodge material that may be blocking the inner lumen.

The inner lumen of the delivery catheter 2625 may also be used for aspiration of emboli or to preferentially facilitate maintaining particles inside the filter 2620 during recapture. The aspirated blood can be re-injected into the patient through an extracorporeal neuroprotection system.

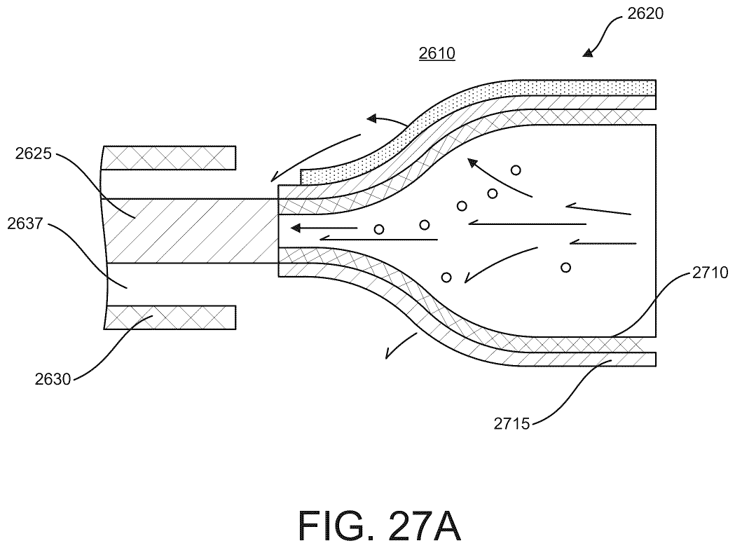

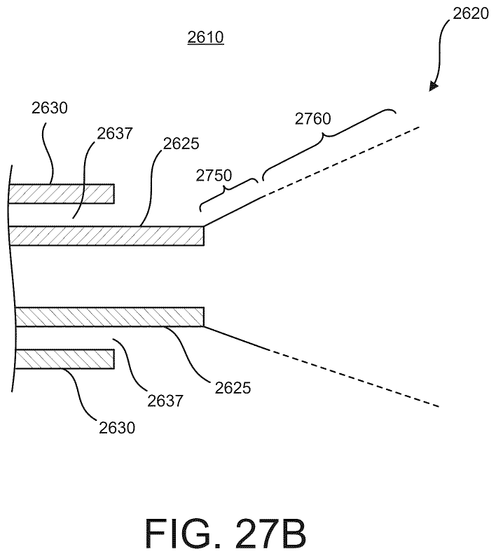

FIG. 27A shows a schematic, side view representation of a distal region of the filter system 2610, which includes the filter 2620. As mentioned, the filter 2620 is shaped with the inner filter layer 2710 positioned within the outer filter layer 2715. The filter 2620 has a funnel shape that funnels into the lumen of the inner delivery catheter 2625. In an example embodiment, the inner filter layer 2710 is a polyurethane film having pores of a smaller size relative to those of the outer filter later 2715. Or the inner filter layer 2710 can have pores of a larger size relative to those of the outer filter later 2715 in an alternate embodiment. The outer filter layer may also be formed of a polyurethane film. In use, blood may pass through pores of a filter membrane and into the inner delivery catheter 2625, which is positioned inside the outer delivery sheath 2630. Blood may also flow through the annular space 2637 between the outer delivery sheath 2630 and the inner delivery catheter 2625. As mentioned, the inner delivery catheter 2625 may be permeable to allow blood flow to the annular space 2637 wherein the blood flows between the inner diameter of the inner delivery catheter 2625 and the inner wall of the outer delivery sheath 2630.

The filter system 2610 is configured to achieve shunting and/or aspiration of blood flow. In this regard, the filter system 2610 is configured to shunt and/or aspirate blood via only the inner delivery catheter 2625, via only the annular space 2637, or via both the inner delivery catheter 2625 and the annular space 2637. Aspiration of blood can be performed via any of these pathways during removal of the filter system 2610 from the vasculature or at any point during use.

FIG. 27B shows another embodiment of the filter system 2610 wherein the filter 2620 includes at least one non-porous region 2750 without any holes such that the filter holes are predisposed toward a distal end of the filter 2620 in the porous region 2760. The filter 2620 further includes a porous region 2760 that includes pores or openings. The porosity of the porous region 2760 can vary. In an embodiment, the porous region 2760 is about 70-95% of total length of filter 2620 although this may vary. The filter 2620 of FIG. 27B is configured to direct a high flow velocity in more narrow regions (such as the funnel-shaped non-porous region 2750) where embolic particles may accumulate. The non-porous region can be a proximal region or a region closest to the inner catheter. The non-porous region can be in a more narrow or constricted region of the filter (such as the more narrow portion of the funnel-shape.)

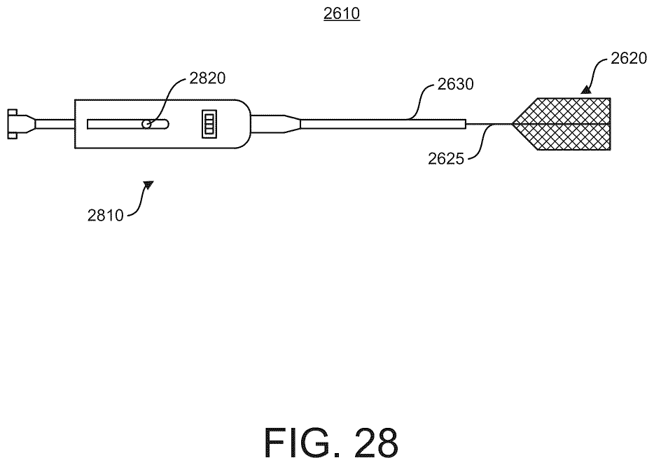

FIG. 28 shows a schematic representation of the filter system 2610. As mentioned, the filter 2620 is mounted on a distal end of the inner delivery catheter 2625, which is movably positioned inside the outer delivery sheath 2630. A control element 2810 is located at a proximal region of the filter system 2610 and is configured to mechanically or electromechanically control the components of the filter system 2610. The inner delivery catheter 2625 may be fixed to the control element 2810 while the outer delivery sheath 2630 may be moved relative to the inner delivery catheter 2625 such as by actuating an actuator 2820 on the control element 2810. In this manner, the outer delivery sheath 2630 may be slidingly moved to capture the filter 2620 within its inner lumen and to also release the filter 2620 such as shown in FIG. 28.

In an embodiment, a user actuates the filter system during use such that the filter 2620 is moved relative to the outer delivery sheath 2630 and/or the inner delivery catheter 2625 in combination with aspiration such as in a back-and-forth manner. Such movement may achieve improved embolic particle retrieval prior to the filter being recaptured in the outer delivery sheath 2630. The filter 2620 may be connected to a wire or other element that enables such movement of the filter 2620.

A pump can be coupled to any of the embodiments described herein to provide active flow of fluid. The pump can be configured to achieve intermittent (or pulsed) flow or continuous flow. Passive blood flow can also be intermittent (or pulsed) flow or continuous flow.

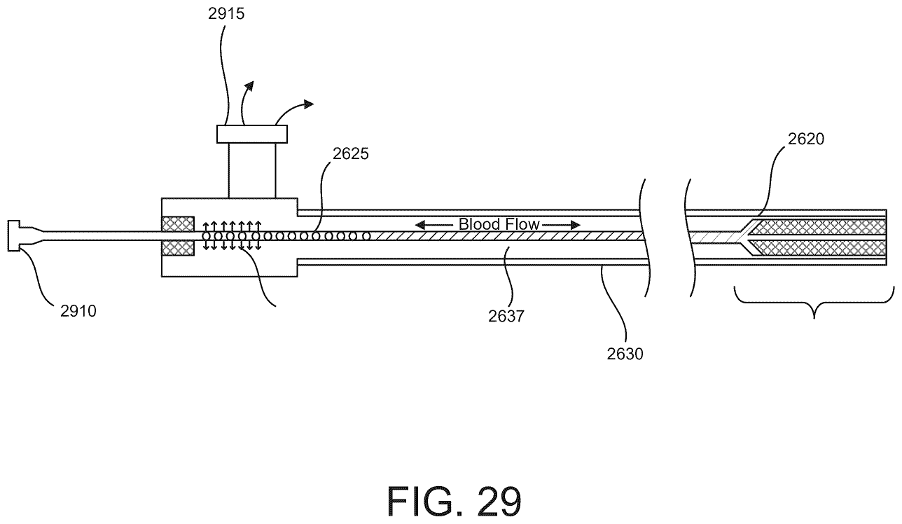

FIG. 29 shows a schematic representation of the filter system 2610 in cross section with the filter 2620 captured and collapsed within the outer delivery sheath 2630. Blood can flow in the annular space 2637 between the outer diameter of the inner delivery catheter 2625 and the inner diameter of the outer delivery sheath 2630. As mentioned, blood flow can transition out of the inner lumen of the inner delivery catheter 2625 into the annular space via small openings in the outer wall of the inner delivery catheter 2625. The inner delivery catheter 2625 can have a proximal hub 2910. The outer delivery sheath 2625 can also have a hub 2915 through which blood can be routed to an artery or vein via a neuroprotection system.

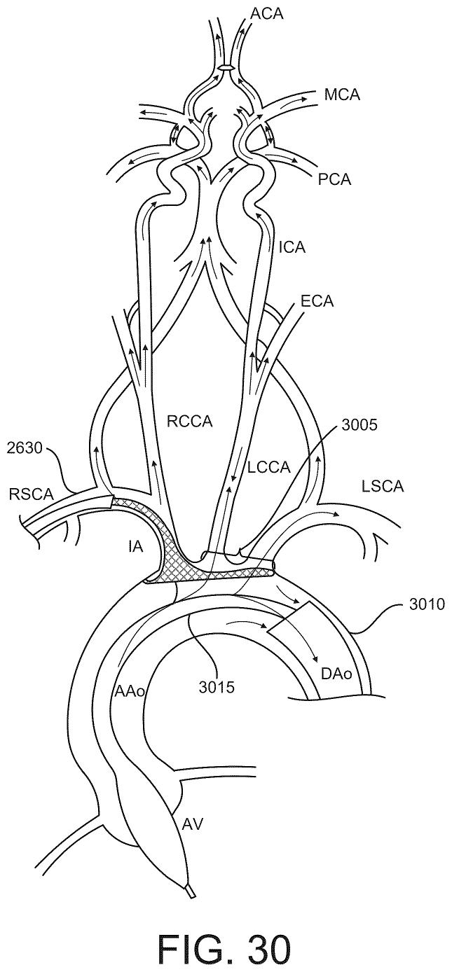

FIG. 30 shows a schematic representation of the vasculature wherein a filter 3005 has been deployed in the aortic arch via a delivery sheath 2630. The filter is positioned to cover openings into the IA, LCCA and LSCA. The sheath 2630 is delivered via a radial approach. The sheath 2630 is configured to provide aspiration flow for capture of debris into the sheath 2630 such as active flow via a pump or passive flow. A second sheath 3010 is delivered to the aortic arch such as via a transfemoral approach. A TAVR device 3015 can be delivered to the aortic arch via a lumen of the second sheath 3010. The TAVR device may trigger the release of embolic particles into the aortic arch. The second sheath 3010 is configured to aspirate and capture embolic particles into its internal lumen via passive or active aspiration flow such as by being coupled to a pump. The tip of the second sheath 3010 may be positioned at various locations along the aortic arch.

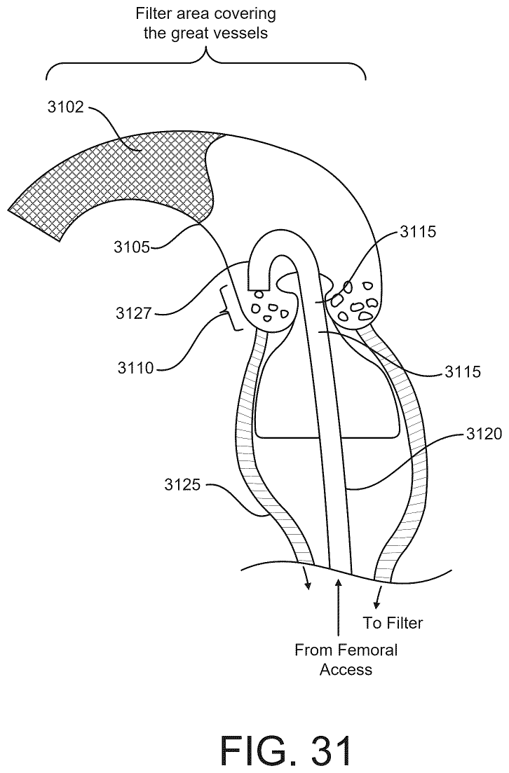

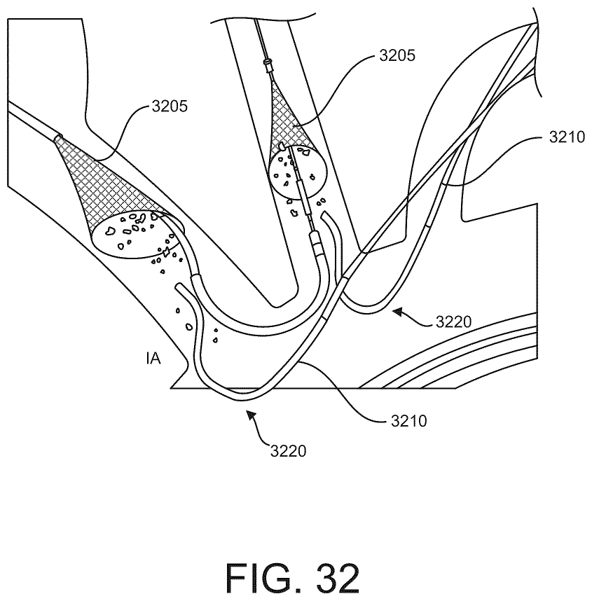

FIG. 31 schematically shows a filter basket 3102, which is deployed to in the aortic arch to filter the area covering the great blood vessels from the aortic arch. The filter basket 3102 is cut away at line 3105 for clarity of illustration. An aspiration catheter 3120 is deployed into the aortic arch with a distal end 3127 positioned and configured to provide aspiration flow to capture embolic debris from the filter basket 3102. The aspiration catheter 3120 has a curved distal tip such as a distal tip that is curved back at about a 180 degree angle to enable the tip to aspirate within a region 3110 where embolic debris is collected/captured. The aspiration catheter 3120 may be delivered via a transfemoral approach for example.

In another embodiment also shown in FIG. 31, at least one catheter 3125 having an internal lumen is coupled to the region 3110 of the filter basket 3102. The catheter(s) 3125 are configured to aspirate material from the filter basket 3102. The catheter 3125 can be configured for constant or intermittent removal of material via active or passive flow.

FIG. 32 shows an embodiment wherein one or more filters 3205 are deployed to the blood vessels branching off of the aortic arch. One or more aspiration catheters 3210 can be deployed to the aortic arch such as via a left radial access or right radial access. The aspiration catheters have curved distal tips 3220 with an opening through which embolic debris can be aspirated from the filters into an internal lumen of the aspiration catheter 3210. The curved distal tip 3220 can have a hook shape that may flare outward at the distal most end such as in a shepherd's hook shape. In an embodiment, the catheters 3210 are 6 French catheters and are connected to a device, such as a syringe or pump, for active aspiration.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A system for treating an artery, comprising:
an outer delivery sheath, the outer delivery sheath configured to be delivered to a vascular location in communication with an aortic arch;
an inner catheter defining an inner lumen, the inner catheter slidably positioned inside the outer delivery sheath such that an outer lumen is formed between an outer wall of the inner catheter and an inner wall of the outer delivery sheath;
a filter positioned at a distal end of the inner catheter, the filter configured to guide debris from a blood vessel into the inner lumen of the inner catheter via blood flow into the filter;
an extracorporeal shunt configured to receive blood flow from the inner lumen; and
a filter coupled to the shunt.

2. The system of claim 1, further comprising a pump coupled to the inner catheter, the pump configured to pump blood into the inner catheter and into the shunt.

3. The system of claim 1, wherein the filter is funnel shaped with a narrow region of the funnel directly attached to the inner catheter.

4. The system of claim 1, wherein the filter is formed of an inner filter layer positioned inside an outer filter layer.

5. The system of claim 4, wherein the filter is coupled to a Nitinol frame.

6. The system of claim 4, wherein the inner filter layer has a set of first pores that are smaller than a set of second pores of the outer filter layer.

7. The system of claim 4, wherein the filter includes at least one non-porous region and a porous region.

8. The system of claim 7, wherein the porous region is about 70-95% of total length of the filter.

9. The system of claim 1, wherein the outer delivery sheath is either (a) a 4 Fr or 5 Fr sheath or (b) a 6 Fr or 7 Fr sheath.

10. The system of claim 1, wherein the shunt communicates with a vein and delivers blood from the inner lumen to the blood vessel.

11. The system of claim 1, wherein the inner lumen is sized to receive a guidewire.

12. The system of claim 1, wherein the outer lumen is an annular space.

13. The system of claim 1, wherein the outer wall of the inner catheter has at least one opening that allows fluid communication from the inner lumen to the outer lumen.

14. The system of claim 13, wherein blood flows simultaneously through the inner lumen and the outer lumen.

15. The system of claim 1, wherein the shunt communicates with both the outer lumen and the inner lumen.

16. The system of claim 1, wherein the filter is configured to be retracted into the inner lumen to cause the filter to collapse to a smaller size.

17. The system of claim 1, wherein the inner catheter has a proximal hub that is fluidly connected to the shunt.

18. The system of claim 1, wherein the blood vessel is the aortic annulus, the common carotid artery, or another blood vessel.

19. The system of claim 1, wherein the filter is an elongated funnel shape configured to cover multiple blood vessels.

20. The system of claim 4, wherein the inner filter layer has a set of first pores that are larger than a set of second pores of the outer filter layer.

21. (canceled)
